(19) United States
(12) Patent Application Publication
(10) Pub. No.: US 2026/0295042 A1
(43) Pub. Date: Oct. 1, 2026

(54) SWITCH RECEPTORS

(71) Applicant: Hoffmann-La Roche Inc., Little Falls, NJ (US)

(72) Inventors: Stephan GASSER, Fislisbach (CH); Christian KLEIN, Bonstetten (CH); Dominic SCHWARZ, Blaubeuren (CH); Dario VENETZ, Bonstetten (CH)

(73) Assignee: Hoffmann-La Roche Inc., Little Falls, NJ (US)

(21) Appl. No.: 19/412,397

(22) Filed: Dec. 8, 2025

Related U.S. Application Data
(63) Continuation of application No. PCT/EP2024/064471, filed on May 27, 2024.

(30) Foreign Application Priority Data
Jun. 6, 2023 (EP) 23177793.9

(51) Int. Cl. A61K 40/11; A61K 40/31; A61K 40/34; A61K 40/35; A61K 40/42; A61P 35/00; A61P 37/04; C07K 14/705; C07K 14/715; C12N 5/0783

(52) U.S. Cl. CPC A61K 40/11; A61K 40/31; A61K 40/34; A61K 40/35; A61K 40/4234; A61P 35/00; A61P 37/04; C07K 14/7051; C07K 14/7155; C12N 5/0636; A61K 2239/21; A61K 2239/24; A61K 2239/28; C07K 2319/03; C07K 2319/43

(57) ABSTRACT

The present disclosure relates to the fields of molecular biology, more specifically cytokine receptors. The present disclosure also relates to methods of medical treatment and prophylaxis, particularly cellular immunotherapy.

### CROSS-REFERENCE TO RELATED APPLICATIONS

This application is a Continuation of International Patent Application No. PCT/EP2024/064471, filed May 27, 2024, which claims the benefit of priority to European Application Number 23177793.9 filed Jun. 6, 2023, each of which is incorporated herein by reference in its entirety.

### SEQUENCE LISTING

This application contains a Sequence Listing which has been submitted electronically in XML format and is hereby incorporated by reference in its entirety. Said XML copy, created on Dec. 8, 2025, is named P38561-US-1_Sequence_Listing_xml and is 88,991 bytes in size.

### FIELD OF THE INVENTION

The present disclosure relates to the fields of molecular biology, more specifically cytokine receptors. The present disclosure also relates to methods of medical treatment and prophylaxis, particularly cellular immunotherapy.

### BACKGROUND

Chimeric antigen receptor (CAR) expressing T cells have shown clinical efficacy in different hematologic malignancies. For solid tumor indications CAR-T cell therapies have yet to provide meaningful clinical benefits over conventional chemo- and immunotherapies. This discrepancy can be attributed to several factors, including tumor heterogeneity, limited immune cell tumor infiltration, the tumor microenvironment (TME) as well as the display or secretion of immune inhibitory factors.

Immune inhibitory ligands such as PD-L1, PD-L2, and CTLA-4, are well known to interact with their respective receptors on T cells, leading to T cell exhaustion and impaired anti-tumor response. Moreover, the release of immune-inhibitory cytokines (e.g., IL10, IL4, TGFβ) can dampen T cell function and promote tumor immune escape.

Interleukin 23 (IL23) has emerged as a potential factor with pro-carcinogenic properties. IL23, produced by tumour-associated immune cells, can promote tumour growth and metastasis (Li et al., Carcinogenesis 34, no. 3 (March 2013): 658-66.; Zhang et al., Carcinogenesis 35, no. 6 (June 2014): 1330-40.; Langowski et al., Nature 442, no. 7101 (July 2006): 461-65.), angiogenesis, mediate tumour immune evasion and suppression (Fu et Al., European Urology 75, no. 5 (May 2019): 752-63.); Teng, et al. Proceedings of the National Academy of Sciences 107, no. 18 (May 4, 2010): 8328-33.), and shows potential as a diagnostic marker for lung- and breast cancer. (Gangemi et al. Journal of Cellular Biochemistry 113, no. 6 (June 2012): 2122-25.); Zhang et al. Therapeutics and Clinical Risk Management Volume 18 (April 2022): 429-37.).

For instance, one study highlights that IL23 blockade in cancer treatment destabilizes intratumoral Tregs and leads to an advantageous outcome (Wight et al., Proceedings of the National Academy of Sciences 119, no. 18 (May 3, 2022): e2200757119.). This is underlined by the fact that IL23 and IL12 are part of the same receptor superfamily, but their signalling consequences can be opposing (Sieve et al., European Journal of Immunology 40, no. 8 (August 2010): 2236-47.; Teng et al., Cancer Research 72, no. 16 (Aug. 15, 2012): 3987-96.; Kortylewski et al., Cancer Cell 15, no. 2 (February 2009): 114-23.).

While the potent antitumor function of IL12 is well documented, approaches to induce IL12 signalling in the tumour remain challenging. For example, a method involving systematic and/or untargeted downregulation of IL-23 or upregulation of IL12 signalling would severely interfere with the proper function of the immune system and have serious consequences are risks for the patient's health. Hence, a strategy that modulates the balance between IL23 and IL12 signalling holds promise in improving CAR-T cell therapy outcomes in solid tumours. On the other hand, while some groups have tried making chimeric receptors (a.k.a. switch receptors) in order to direct the signalling pathway to the desired route, one of the challenges associated with this strategy is finding mutations that efficiently prevent activation by the original ligand without otherwise compromising the receptor's structure or intrinsic ability to activate gene transcription.

The disclosure provided here provides solutions to the problems existing with previous attempts to manipulate immune cells and potentially offer improved methods for treatments involving cell transfer.

### SUMMARY OF THE INVENTION

The present invention generally relates to chimeric cytokine receptors (also referred to as switch receptors or chimeric receptor polypeptides herein) capable of translating an interleukin-23 signal into a cellular response akin to interleukin-12. The chimeric cytokine receptors of the invention are comprised of at least the first extracellular domain of the interleukin-23 receptor and further extracellular domains of the interleukin-12 receptor beta 2, the intracellular domains of the Interleukin-12 receptor beta 2 and the transmembrane domain of either receptor. After engagement of the chimeric cytokine receptor with interleukin-23 and interleukin-12 receptor beta 1, a chimeric receptor complex is formed that leads to an intracellular response akin to interleukin-12, in a dose-dependent manner. The invention also relates to nucleic acid molecules encoding the chimeric cytokine receptors of the present invention, vectors, cells, pharmaceutical compositions, and uses and methods as described below.

In a first aspect, the present disclosure provides a chimeric receptor polypeptide comprising:
(i) an extracellular portion comprising an IL23-binding domain;
(ii) an intracellular portion comprising the intracellular domains of IL12Rb2 (Interleukin-12 receptor beta 2); and
(iii) a transmembrane domain that joins the extracellular portion and the intracellular portion.

In some embodiments, the IL23-binding domain of the chimeric receptor polypeptide binds the p19 subunit of IL23.

In some embodiments, the intracellular portion comprises the amino acid sequence of SEQ ID NO: 5 or an amino acid sequence having at least 80% (preferably one of ≥80%, ≥85%, ≥90%, ≥91%, ≥92%, ≥93%, ≥94%, ≥95%, ≥96%, ≥97%, ≥98%, ≥99%) sequence identity thereto.

In some embodiments, the transmembrane domain is selected from the transmembrane domain of IL12Rb2, IL23Ra, CD8, and CD28.

In some embodiments, the transmembrane domain comprises an amino acid sequence selected from SEQ ID Nos: 3, 4, 22, 33, or an amino acid sequence having at least 80% (preferably one of ≥80%, ≥85%, ≥90%, ≥91%, ≥92%, ≥93%, ≥94%, ≥95%, ≥96%, ≥97%, ≥98%, ≥99%) sequence identity thereto.

In some embodiments, the chimeric receptor polypeptide further comprises a signal sequence.

In some embodiments, the signal sequence comprises the amino acid sequence of SEQ ID NO: 1 or 16.

In some embodiments, the chimeric receptor polypeptide further comprises one or more linkers.

In some embodiments, the linker is a GS linker, a 2A linker, an □-helical linker, a glycine-alanine polymer linker, an alanine-serine polymer linker, or an IgG4-Fc linker.

In some embodiments, the chimeric receptor polypeptide further comprises a tag polypeptide, e.g. a Flag tag.

In some embodiments, the chimeric receptor polypeptide further comprises a marker, e.g. a fluorescent polypeptide, e.g. GFP or eGFP.

In some embodiments, the chimeric receptor polypeptide comprises from the N-terminus to the C-terminus:

Optional signal peptide-optional tag-optional linker-IL23-binding domain-transmembrane domain-intracellular portion comprising the intracellular domains of IL12Rb2-optional linker-optional marker

In some embodiments, the IL23-binding domain comprises an extracellular domain of IL23R (a.k.a. IL23R).

In some embodiments, the IL23-binding domain comprises the amino acid sequence of SEQ ID NO: 10 or an amino acid sequence having at least 80% (preferably one of ≥80%, ≥85%, ≥90%, ≥91%, ≥92%, ≥93%, ≥94%, ≥95%, ≥96%, ≥97%, ≥98%, ≥99%) sequence identity thereto.

In some embodiments, the IL23-binding domain comprises the amino acid sequence of SEQ ID NO: 35 or an amino acid sequence having at least 80% (preferably one of ≥80%, ≥85%, ≥90%, ≥91%, ≥92%, ≥93%, ≥94%, ≥95%, ≥96%, ≥97%, ≥98%, ≥99%) sequence identity thereto.

In some embodiments, the IL23-binding domain comprises the amino acid sequence of SEQ ID NO: 2 or an amino acid sequence having at least 80% (preferably one of ≥80%, ≥85%, ≥90%, ≥91%, ≥92%, ≥93%, ≥94%, ≥95%, ≥96%, ≥97%, ≥98%, ≥99%) sequence identity thereto.

In some embodiments, the chimeric receptor polypeptide further comprises, between the IL23-binding domain and the transmembrane domain, an amino acid sequence of SEQ ID NO: 47 or an amino acid sequence having at least 80% (preferably one of ≥80%, ≥85%, ≥90%, ≥91%, ≥92%, ≥93%, ≥94%, ≥95%, ≥96%, ≥97%, ≥98%, ≥99%) sequence identity thereto.

In some embodiments, the chimeric receptor polypeptide further comprises, between the IL23-binding domain and the transmembrane domain, an amino acid sequence of SEQ ID NO: 48 or an amino acid sequence having at least 80% (preferably one of ≥80%, ≥85%, ≥90%, ≥91%, ≥92%, ≥93%, ≥94%, ≥95%, ≥96%, ≥97%, ≥98%, ≥99%) sequence identity thereto.

In some embodiments, the chimeric receptor polypeptide further comprises, between the IL23-binding domain and the transmembrane domain, an amino acid sequence of SEQ ID NO: 15 or an amino acid sequence having at least 80% (preferably one of ≥80%, ≥85%, ≥90%, ≥91%, ≥92%, ≥93%, ≥94%, ≥95%, ≥96%, ≥97%, ≥98%, ≥99%) sequence identity thereto.

In some embodiments, the IL23-binding domain comprises a Fv, scFv, Fab, Fab′, Fab′-SH, F(ab′)2, crossFab, scFab, a single domain antibody (sdAb), or a designed ankyrin repeat protein (DARPin).

In some embodiments, the chimeric receptor polypeptide comprises an amino acid sequence having at least 80% (preferably one of ≥80%, ≥85%, ≥90%, ≥91%, ≥92%, ≥93%, ≥94%, ≥95%, ≥96%, ≥97%, ≥98%, ≥99%) sequence identity to an amino acid sequence selected from SEQ ID Nos: 36-44.

In a second aspect, the present disclosure provides a nucleic acid, or a plurality of nucleic acids, encoding a chimeric receptor polypeptide disclosed herein, e.g. in the first aspect and the embodiments thereof, and optionally further comprising a nucleic acid sequence encoding a chimeric antigen receptor.

In a third aspect, the present disclosure provides an expression vector, or a plurality of expression vectors, comprising a nucleic acid or a plurality of nucleic acids disclosed herein, e.g. in the second aspect.

In a fourth aspect, the present disclosure provides a cell comprising a chimeric receptor polypeptide according to the first aspect, a nucleic acid or a plurality of nucleic acids according to the second aspect, or an expression vector or a plurality of expression vectors according to the third aspect.

In some embodiments, the cell expresses IL12Rb1 (Interleukin-12 receptor beta 1 chain).

In some embodiments, the cell is a eukaryotic cell. In some embodiments, the eukaryotic cell is an animal cell. In some embodiments, the animal cell is a mammalian cell. In some embodiments, the mammalian cell is a human cell.

In some embodiments, the cell is an immune cell, a neuron, an epithelial cell, an endothelial cell, or a stem cell.

In some embodiments, the cell is an immune cell.

In some embodiments, the immune cell is a tumor infiltrating lymphocyte (TIL), B cell, a monocyte, a natural killer (NK) cell, a basophil, an eosinophil, a neutrophil, a dendritic cell, a macrophage, a regulatory T cell (Treg), a helper T cell (Th), a cytotoxic T cell (Tctl), an effector T cell, a memory T cell, a Natural Killer T (NKT) cell, or other T cell. In some embodiments, the immune cell further comprises a chimeric antigen receptor (CAR). In some embodiments, the immune cell further comprises an engineered TCR.

In some embodiments, the cell shows a dose-dependent phosphorylation of STAT4 but not STAT3 when treated with IL23.

In some embodiments, the cell is a transduced T cell capable of expressing the chimeric receptor polypeptide according to the first aspect and the embodiments thereof.

In some embodiments, the cell expresses an antigen binding receptor comprising an anchoring transmembrane domain and an extracellular domain, wherein the extracellular domain comprises an antigen binding moiety comprising
(i) a heavy chain variable domain (VH) comprising a heavy chain complementary determining region (HCDR) 1 of SEQ ID NO:49, a HCDR 2 of SEQ ID NO:50 or SEQ ID NO:51, and a HCDR 3 of SEQ ID NO:52, and
(ii) a light chain variable domain (VL) comprising a light chain complementarity determining region (LCDR) 1 of SEQ ID NO:53, a LCDR 2 of SEQ ID NO:54 and a LCDR 3 of SEQ ID NO:55.

In some embodiments, the VH domain comprises an amino acid sequence that is at least about 95%, 96%, 97%, 98%, 99% or 100% identical to an amino acid sequence selected from the group consisting of SEQ ID NO:56, SEQ ID NO:57, SEQ ID NO:58 and SEQ ID NO 59.

In some embodiments, the VL domain comprises an amino acid sequence that is at least about 95%, 96%, 97%, 98%, 99% or 100% identical to the amino acid sequence of SEQ ID NO:60 or SEQ ID NO:61.

In some embodiments, the anchoring transmembrane domain is a transmembrane domain selected from the group consisting of the CD8, the CD4, the CD3z, the FCGR3A, the NKG2D, the CD27, the CD28, the CD137, the OX40, the ICOS, the DAP10 or the DAP12 transmembrane domain or a fragment thereof, in particular wherein the anchoring transmembrane domain is the CD8 transmembrane domain or a fragment thereof.

In some embodiments, the antigen binding receptor further comprises at least one stimulatory signalling domain and/or at least one co-stimulatory signalling domain.

In some embodiments, the antigen binding receptor at least one stimulatory signalling domain is individually selected from the group consisting of the intracellular domain of CD3z, of FCGR3A and of NKG2D, or fragments thereof that retains stimulatory signalling activity, in particular wherein the at least one stimulatory signalling domain is the CD3z intracellular domain or a fragment thereof that retains CD3z stimulatory signalling activity.

In some embodiments, the antigen binding receptor the at least one co-stimulatory signalling domain is individually selected from the group consisting of the intracellular domain of CD27, of CD28, of CD137, of OX40, of ICOS, of DAP10 and of DAP12, or fragments thereof that retain co-stimulatory signalling activity.

In some embodiments, the antigen binding receptor comprises at least one CD28 costimulatory domain or a fragment thereof that retains CD28 co-stimulatory activity, and/or at least one CD137 costimulatory domain or a fragment thereof that retains CD137 co-stimulatory activity.

In some embodiments, the antigen binding receptor comprises a stimulatory signalling domain comprising the intracellular domain of CD3z, or a fragment thereof that retains CD3z stimulatory signalling activity, and wherein the antigen binding receptor comprises a co-stimulatory signalling domain comprising the intracellular domain of CD28, or a fragment thereof that retains CD28 co-stimulatory signalling activity.

In some embodiments, the antigen binding receptor comprises one stimulatory signalling domain comprising the intracellular domain of CD3z, or a fragment thereof that retains CD3z stimulatory signalling activity, and wherein the antigen binding receptor comprises one co-stimulatory signalling domain comprising the intracellular domain of CD137, or a fragment thereof that retains CD137 co-stimulatory signalling activity.

In some embodiments, the antigen binding moiety is connected at the C-terminus to the N-terminus of the anchoring transmembrane domain, optionally through a peptide linker.

In some embodiments, in the antigen binding receptor the light chain variable domain (VL) of the antigen binding moiety is connected at the C-terminus to the N-terminus of the anchoring transmembrane domain, optionally through a peptide linker, and/or wherein the heavy chain variable domain (VH) is connected at the C-terminus to the N-terminus of the light chain variable domain (VL), optionally through a peptide linker.

In a fifth aspect, the present disclosure provides a pharmaceutical composition comprising the cell according to the fourth aspect, and optionally a pharmaceutically acceptable excipient.

In a fifth aspect, the present disclosure provides the chimeric receptor polypeptide according to the first aspect, or the nucleic acid(s) according to the second aspect, or the cell according to the third aspect, or the pharmaceutical composition according to the fourth aspect, for use in a method of medical treatment of a disease or prophylaxis of a disease. In some embodiments, the method is a cell therapy, e.g. an adoptive cell therapy, the disease is a cancer, an autoimmune disease, or an infection. In some embodiments, the cancer is selected from the group consisting of acute leukemias (including but not limited to acute myeloid leukemia (AML), B-cell acute lymphoid leukemia (BALL), T-cell acute lymphoid leukemia (TALL), and acute lymphoid leukemia (ALL)), chronic leukemias (including but not limited to chronic myelogenous leukemia (CML) and chronic lymphocytic leukemia (CLL)), multiple myeloma (MM), myelodysplastic syndrome (MDS), myeloproliferative neoplasms (MPNs), chronic myeloid leukemia (CML), and blastic plasmacytoid dendritic cell neoplasm (BPDCN).

In some embodiments, the chimeric receptor polypeptide according to the first aspect, or the nucleic acid(s) according to the second aspect, or the cell according to the third aspect, or the pharmaceutical composition according to the fourth aspect, is administered intravenously, intratumorally, or subcutaneously.

In a sixth aspect, the present disclosure provides a method for modulating the activity of an immune cell, said method comprising: administering, to an immune cell, the nucleic acid or plurality of nucleic acids according to the second aspect, or the expression vector or plurality of expression according to the third aspect. In some embodiments, the immune cell is a tumour infiltrating lymphocyte (TIL), B cell, a monocyte, a natural killer (NK) cell, a basophil, an eosinophil, a neutrophil, a dendritic cell, a macrophage, a regulatory T cell (Treg), a helper T cell (Th), a cytotoxic T cell (Tctl), an effector T cell, a memory T cell, a Natural Killer T (NKT) cell, or other T cell.

### BRIEF DESCRIPTION OF THE FIGURES

Embodiments and experiments illustrating the principles of the present disclosure will now be discussed with reference to the accompanying Figures.

FIG. 1A. Scientific rationale for the design of IL23-IL12sRs for adoptive cell therapy. A) Heatmap describing the distribution of IL23 in tumour- and normal tissues compiled from >11,000 RNA-seq samples available from the Cancer Genome Atlas (TCGA) database. Several tumours, dominantly lung and colon, display transcriptomic evidence of overexpression of IL23A in the tumour compared to normal tissue. B) Immunohistochemistry staining of HPAF-II (left) and MKN-45 (right) tumours in xenograft mouse models show presence of the protein in epithelial parts of the tumour.

FIG. 1B. Schematic representation of wild type IL12 and IL23 receptor complexes and chimeric IL23-IL12 switch Receptors (IL23-IL12sR).

FIG. 1C. Depictions of alternative IL23-IL12sR formats.

FIG. 1D. Overview of expression cassettes of IL23-IL12sR formats in a screening plasmid combined with a downstream GFP reporter.

FIG. 1E. Overview of expression cassettes of IL23-IL12sR formats as downstream gene element of different CAR constructs including variants with chimeric ECDs. AB-SP: human IgG1 antibody signal peptide; Tag: FLAG epitope tag. E2A can be replaced by another 2A self-cleaving peptide, e.g. T2A.

FIG. 1F. Depiction of the mechanism of action of an immune cell engineered with IL23-IL12sRs.

FIGS. 2A-2B. Detection of IL23-IL12sR on engineered immune cells. FIG. 2A. The expression of engineered IL23-IL12sRs was assessed indirectly by the expression of eGFP, which is placed downstream of the IL23-IL12sR expression cassette separated by a self-cleaving 2A-peptide. FIG. 2B. Detection of the anti P329G-CAR via AF647-labeled Fc molecule with P329G mutation and a FLAG-tagged variant of the IL23-IL12sR

FIGS. 3A-3D. T cells engineered with an IL23-IL12sR show dose-dependent phosphorylation of [[A)]] STAT4 (FIG. 3A) but not STAT3 (FIG. 3B) in response to IL23 stimulation. Data in flow cytometric analysis is gated on lymphocytes and single cells (both FSC and SSC). IL23-IL12sR T cells are additionally gated on GFP reporter fluorescence. FIG. 3C. The upregulation of STAT4 can also be observed in T cells engineered with a P329G CAR and IL23-IL12sR. FIG. 3D. Upregulation of STAT3 in response to IL23 stimulation can only be observed in T cells overexpressing a recombinant wild type IL23R in vitro.

FIG. 4. Functional analysis of T cells engineered with an IL23-IL12sR via detection of alternative surface expression of IL12 signalling-related markers in flow cytometry. A) Most prominently, the IL18Ra is upregulated in response to IL23 stimulation, in engineered cells exclusively. The response of an IL23-IL12sR variant with IL12Rb2-TMD appears 10-fold more sensitive. Either response shows a lower EC50 than stimulation of wild type cells by IL12. B) In IL23-IL12sR T cells exclusively, the upregulation of IL12Rb2 via a feed-forward mechanism triggered by STAT4 phosphorylation can be observed. Here, IL23-IL12sR (IL12Rb2-TMD) T cells show a lower IL12Rb2-baseline, more similar to the wild type cells, when compared to IL23-IL12sR (IL23R-TMD).

FIG. 5. Functional analysis of T cells engineered with a P329G-CAR with 41BB costimulation and IL23-IL12sR via detection of alternative surface expression of IL12 signalling-related markers in flow cytometry. A) Compared to previous results without co-expression of a P329G CAR, the IL18Ra upregulation is 2-fold higher, compared to wild type T cells stimulated with IL12. B) Upregulation of IL12Rb2 via a feed-forward mechanism triggered by STAT4 phosphorylation can be observed as well. C) Secretion of IFNγ can be observed in a dose-dependent manner in response to IL23 or IL12. D) Compared to wild type T cells, the IFNγ release mediated by IL12 is increased. A similar response is also triggered via stimulation with IL23 in cells with a P329G-CAR and IL23-IL12sR, but not in cells engineered with an IL23-IL12sR only.

FIG. 6. Immune cells engineered with a P329G-CAR and FLAG-tagged IL23-IL12sR show increased killing of MKN-45 and HPAF-II target cells when stimulated with IL23. A) Engineered cells titrated with CEA-CAMS targeted IgG antibody carrying a P329G mutation in the Fc portion (e.g. as disclosed in WO2022029051A1) show dose-dependent killing efficiency in vitro. At suboptimal IgG adaptor concentrations, the addition of IL23 triggering IL23-IL12sRs potentiates target cell growth inhibition, represented as 20-40% increased MKN-45 killing in vitro. B) Likewise, CEACAM5 targeted killing of HPAF-II target cells is improved by 10-20%.

FIGS. 7A-7B. Detection and functional characteristics of immune cells engineered with a CEA-CAMS CAR and non-FLAG-tagged IL23-IL12sR linked with a self-cleaving 2A-peptide. FIG. 7A. anti-CEA-CAM5 CAR expression was assessed with biotinylated CEA-Fc fusion molecule and secondary staining via AF647-labeled Streptavidin. IL23-IL12sR cannot be detected but expected to be similar to the CAR, analogous to previous results. FIG. 7B. T cells engineered with a direct CEA-CAR and IL23-IL12sR show dose dependent upregulation of surface markers IL18Ra and IL12Rb2, CD25 as well as secretion of IFNg. Compared to wild type T cells, the baseline CD25 level is increased.

FIGS. 8A-8B. Immune cells engineered with a CEA-CAR and IL23-IL12sR show increased killing of MKN-45 target cells when stimulated with IL23. FIG. 8A. Especially in conditions with low effector to target (E:T) ratio, the effect is most apparent, ranging from an increase of 10-40% in overall growth inhibition, represented as cell killing. Notably, the increase in killing capacity induced by either IL12 at 100 ng/ml or IL23 at 100 or 1000 ng/mL is very similar. FIG. 8B. Summary of killing capacity plotted as a function of ET ratio, extracted on day 2 of the experiment.

FIGS. 9A-9B. Alternative IL23-IL12sR formats and functional characteristics thereof. FIG. 9A. The expression of alternative IL23-IL12sR formats can be detected indirectly by its effect on P329G-CAR expression, and directly by detection of the N-terminal FLAG-tag. Notably, full IL23R-ECD IL23-IL12sR variants express best and all variants with chimeric ECD influence the expression of the P329G-CAR negatively. FIG. 9B. All IL23-IL12sR formats lead to a similar upregulation of the IL18Ra, IL12Rb2 and IFNg release. Across all readouts, in IL23-IL12sR (1+5) and (3+3 variants, the dose-response is shifted to the left, and overall signal is increased.

FIG. 10. Alternative IL23-IL12sR formats and resulting increased CAR-T cell killing thereof. All variants except the IL23-IL12sR with IL23R-TMD show increased T cell killing when stimulated with IL23. Notably, variants with IL12Rb2 TMD and variants with (1+5) chimeric ECD show best killing without IL23 stimulation. Variants with (1+5) or (3+3) chimeric ECD show highest response to IL23 across different E:T ratios.

FIG. 11. Immune cells engineered with a CEA-CAR and IL23-IL12sR and stimulated with IL23 show persistent killing of HPAF-II target cells in a repetitive killing assay. Effector and target cells were seeded at a ratio of 1:1. Effector cells were transferred to fresh target cells every 2 days with or without the addition of cytokines. Both CEA-CAR T cells and CEA-CAR IL23-IL12sR T cells control the growth of HPAF-II cells CEA-CAR IL23-IL12sR T cells show repeated killing of target cells across 5 rounds.

### DETAILED DESCRIPTION

Chimeric antigen receptor (CAR) expressing T cells have shown remarkable clinical efficacy, particularly improving survival rates of patients with advanced haematological malignancies. However, inhibitory factors of the tumour microenvironment, such as checkpoint molecules and immunosuppressive cytokines, limit CAR-T cell function particularly targeting solid cancers thus far. One approach of manipulating the tumour microenvironment used in the present invention is the development of switch receptors that translate an inhibitory signal into a beneficial signal.

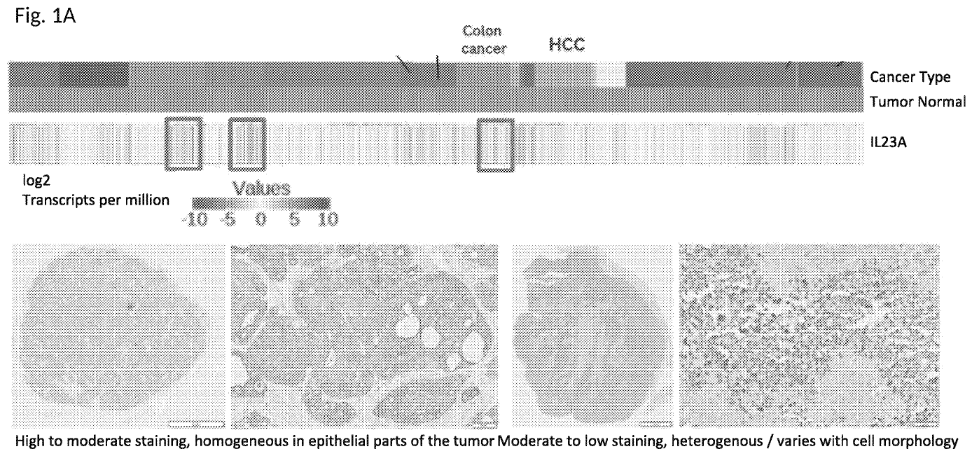

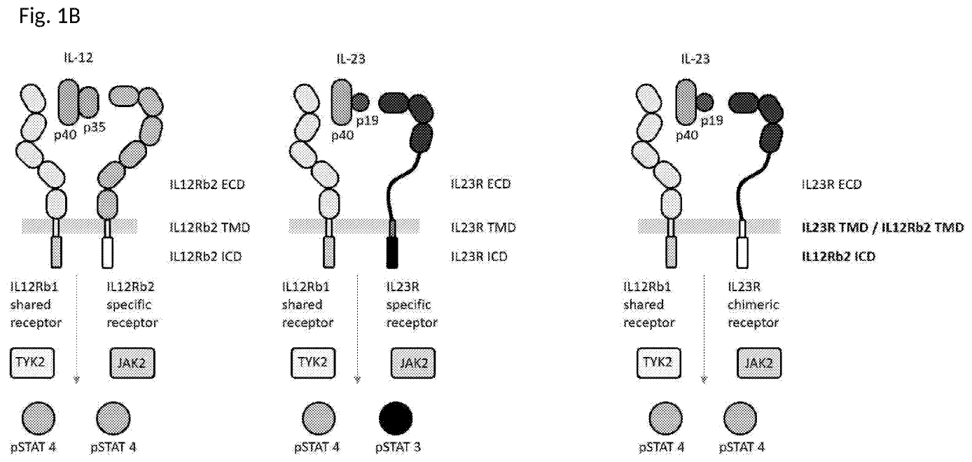

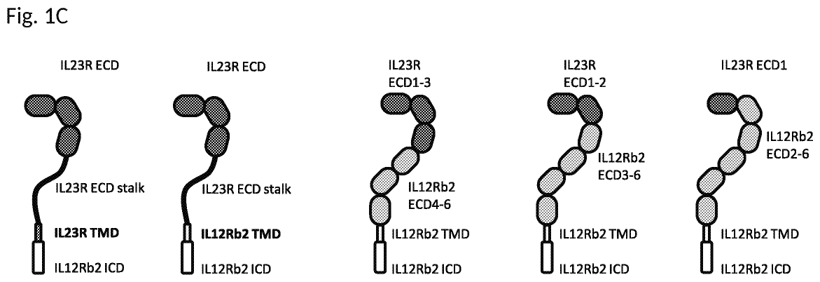

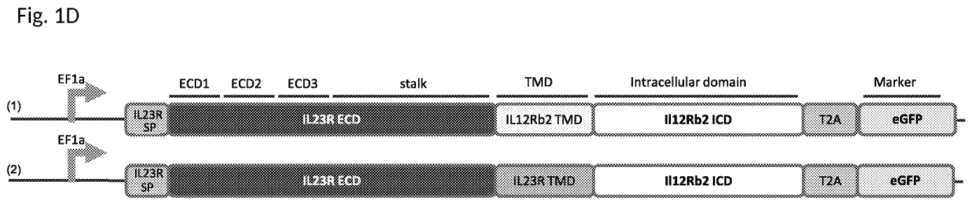

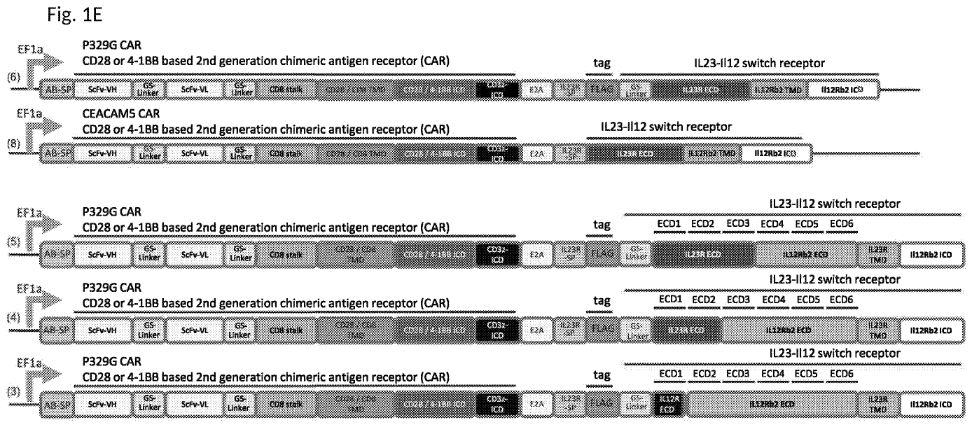

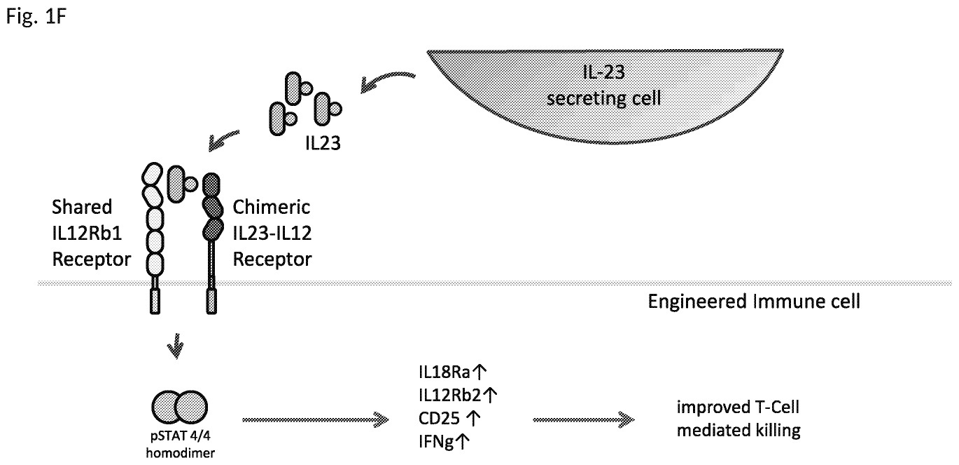

We found out that several tumours, dominantly lung and colon, display transcriptomic evidence of overexpression of IL23A in the tumour compared to normal tissue (FIG. 1A). Here, we make use of the evolutionary relationship of the IL23 and IL12 pathways and their shared receptor chain, IL12Rb1. Indeed, as indicated in FIG. 1B, both complexes share the IL12Rb1 via their common p40 cytokine subunit. IL12Rb1 is constitutively expressed on the surface of human immune cells. IL12 specifically interacts with the IL12Rb2 via its p35 subunit to promote TYK2 and JAK2-mediated phosphorylation of STAT4. IL23 specifically interacts with the IL23R to promote TYK2 and JAK2-mediated phosphorylation of STAT4 and STAT3. Chimeric IL23-IL12 switch receptors are composed of extracellular domains (ECD) of IL23R and intracellular domain (ICD) of IL12Rb2. Together with IL12Rb1, IL23R-IL12Rb2 switch receptors induce IL12-like signalling via phosphorylation of STAT4 upon stimulation with IL23.

The chimeric switch receptors of the present invention translate the pro-carcinogenic signal of IL23 into a cytotoxic IL12 signal. Surprisingly, T cells, e.g. CAR-T cells, engineered with these switch receptors differentially regulate important surface markers and secreted factors in response to stimulation and show increased cancer cell line killing in vitro.

Alternative IL23-IL12sR (sR: Switch Receptor) formats are indicated in FIG. 1C. IL23 is bound by the IL23R_ECD1 domain. All chimeric IL23R-IL12Rb1 switch receptors contain the IL23R_ECD1 and C-terminal IL23R ECDs were replaced by the corresponding number of IL12Rb2 ECDs resulting in different chimeric IL23R-IL12Rb2 designs.

An overview of expression cassettes of IL23-IL12sR formats in a screening plasmid combined with a downstream GFP reporter is shown in FIG. 1D. Constructs (1) and (2) correspond to SEQ ID Nos: 36 and 37, respectively.

An overview of expression cassettes of IL23-IL12sR formats as downstream gene element of different CAR constructs including variants with chimeric ECDs is indicated in FIG. 1E. Constructs (3), (4), (5), (6) and (8) correspond to SEQ ID Nos: 38, 39, 40, 41 and 42, respectively.

The mechanism of action of an immune cell engineered with IL23-IL12sRs is shown in FIG. 1F. IL23 is released by cells near engineered immune cells, e.g., in the tumour microenvironment. IL23 is recognized by chimeric IL23-IL12sR and the endogenous IL12Rb1 leading to formation of the chimeric IL23-IL12sR complex and phosphorylation of STAT4. STAT4 signalling leads to the upregulation of IL18Ra, IL12Rb2, CD25 and secretion of IFNγ. These effects combined can lead to improved killing of target cells in vitro by IL23-IL12sR engineered immune cells.

In the following detailed description, the illustrative alternatives described in the detailed description and claims are not meant to be limiting. Other alternatives may be used and other changes may be made without departing from the spirit or scope of the subject matter presented here. It will be readily understood that the aspects, as generally described herein, can be arranged, substituted, combined, and designed in a wide variety of different configurations, all of which are explicitly contemplated and make part of this application.

### Definitions

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

The terms “administration” and “administering”, as used herein, refer to the delivery of a composition or formulation as disclosed herein by an administration route including, but not limited to, intravenous, intra-arterial, intracranial, intramuscular, intraperitoneal, subcutaneous, intramuscular, or combinations thereof. The term includes, but is not limited to, administration by a medical professional and self-administration.

“Cancer” refers to the presence of cells possessing several characteristics typical of cancer-causing cells, such as uncontrolled proliferation, immortality, metastatic potential, rapid growth and proliferation rate, and certain characteristic morphological features. Cancer cells can aggregate into a mass, such as a tumour, or can exist alone within a subject. A tumour can be a solid tumour, a soft tissue tumour, or a metastatic lesion. As used herein, the term “cancer” also encompasses other types of non-tumour cancers. Non-limiting examples include blood cancers or hematological cancers, such as leukemia. Cancer can include premalignant, as well as malignant cancers.

The terms “cell”, “cell culture”, and “cell line” refer not only to the particular subject cell or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutations (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

The term “percent identity”, as used herein in the context of two or more nucleic acids or proteins, refers to two or more sequences or sub-sequences that are the same or have a specified percentage of nucleotides or amino acids that are the same (e.g., about 80% sequence identity, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the complement of a sequence. This definition also includes sequences that have deletions and/or additions, as well as those that have substitutions. Sequence identity can be calculated over a region that is at least about 20 amino acids or nucleotides in length, or over a region that is 10-100 amino acids or nucleotides in length, or over the entire length of a given sequence. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

As used herein, a “subject” or an “individual” includes animals, such as human (e.g., human subject) and non-human animals. In some embodiments, a “subject” or “individual” is a patient under the care of a physician. Thus, the subject can be a human patient or a subject who has, is at risk of having, or is suspected of having a disease of interest (e.g., cancer) and/or one or more symptoms of the disease. The subject can also be a subject who is diagnosed with a risk of the condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, etc. As a non-limiting example, each range discussed herein can be readily broken down into a lower third, middle third and upper third, and so forth. As will also be understood by one skilled in the art all language such as “up to”, “at least”, “greater than”, “less than”, and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Chimeric Receptor Polypeptides

The present disclosure is based, inter alia, on chimeric receptor polypeptides (a.k.a. chimeric receptors or switch receptors) which comprise an extracellular portion comprising an IL23-binding domain, an intracellular portion comprising the intracellular domains of IL12Rb2, and a transmembrane domain that joins the extracellular portion and the intracellular portion. Immune cells expressing these chimeric receptors may be useful in the context of modulating immune cell activity. In some embodiments, the ligand can be added exogenously and not be limited to production within the cell.

The present disclosure also relates to nucleic acid molecules encoding the chimeric cytokine receptors of the present invention, vectors, cells, pharmaceutical compositions, and uses and methods, as described herein, e.g. in the claims.

### Antigen-Binding Moieties

In some embodiments, the cell expressing the chimeric receptor polypeptide of the invention also expresses an antigen binding receptor comprising an anchoring transmembrane domain and an extracellular domain.

As used herein, an ‘antigen-binding moiety’ refers to a moiety that binds to a given target antigen.

Antigen-binding moieties include antibodies (i.e. immunoglobulins (Igs)), and antigen-binding fragments and derivatives thereof. In some embodiments, an antigen-binding moiety according to the present disclosure comprises, or consists of, a monoclonal antibody, a monospecific antibody, a multispecific (e.g., bispecific, trispecific, etc.) antibody, a variable fragment (Fv) moiety, a single-chain Fv (scFv) moiety, a fragment antigen-binding (Fab) moiety, a single-chain Fab moiety (scFab), a crossFab moiety, a Fab′ moiety, a Fab′-SH moiety, a F(ab′) 2 moiety, a diabody moiety, a triabody moiety, an scFv-Fc moiety, a minibody moiety, a heavy chain only antibody (HCAb) moiety, or a single domain antibody (dAb, VHH) moiety.

Antigen-binding moieties according to the present disclosure also include further target antigen-binding peptides/polypeptides such as peptide aptamers, thioredoxins, anticalins, Kunitz domains, avimers, knottins, fynomers, atrimers, DARPins, affibodys, affilins, armadillo repeat proteins (ArmRPs), OBodys and adnectins (reviewed e.g. in Reverdatto et al., Curr Top Med Chem. 2015; 15(12): 1082-1101, which is hereby incorporated by reference in its entirety (see also e.g. Boersma et al., J Biol Chem (2011) 286:41273-85 and Emanuel et al., Mabs (2011) 3:38-48)). Antigen-binding moieties according to the present disclosure also include target antigen-binding nucleic acids, e.g. nucleic acid aptamers (reviewed, for example, in Zhou and Rossi Nat Rev Drug Discov. 2017 16 (3): 181-202). Antigen-binding moieties according to the present disclosure also include target antigen-binding small molecules (e.g. low molecular weight (<1000 daltons, typically between ~300-700 daltons) organic compounds).

In some embodiments, the antigen-binding moieties are capable of binding to a variant Fc domain, e.g. as disclosed in WO2022029051A1, the contents of which is incorporated herein by reference in its entirety. The antigen-binding moieties described herein preferably display specific binding to a variant Fc domain e.g. as disclosed in WO2022029051A1. As used herein, ‘specific binding’ refers to binding which is selective for the target antigen, and which can be discriminated from non-specific binding to non-target antigen. An antigen-binding moiety that specifically binds to a given target antigen preferably binds the target antigen with greater affinity, and/or with greater duration than it binds to other, non-target antigens.

The ability of a given moiety to bind specifically to a given target antigen can be determined by analysis according to methods known in the art, such as by ELISA, Surface Plasmon Resonance (SPR; see e.g. Hearty et al., Methods Mol Biol (2012) 907:411-442), Bio-Layer Interferometry (BLI; see e.g. Lad et al., (2015) J Biomol Screen 20 (4): 498-507), flow cytometry, or by a radiolabeled antigen-binding assay (RIA) enzyme-linked immunosorbent assay. Through such analysis binding to a given target antigen can be measured and quantified. In some embodiments, the level of binding may be the response detected in a given assay.

An antigen-binding moiety that ‘does not bind’ or that ‘displays substantially no binding’ to a given antigen displays a level of binding to the given antigen which is similar to the level of binding to an antigen that the antigen-binding moiety is known not to bind, or known to not to bind specifically, e.g. a non-target antigen. In some embodiments, the level of binding of an antigen-binding moiety that does not bind, or that displays substantially no binding, to a given antigen is ≥0.5 times and ≤2 times, e.g. one of ≥0.75 times and ≤1.5 times, ≥0.8 times and ≤1.4 times, ≥0.85 times and ≤1.3 times, ≥0.9 times and ≤1.2 times, ≥0.95 times and ≤1.1 times the level of binding displayed by the antigen-binding moiety to an antigen that the antigen-binding moiety is known not to bind, or known to not to bind specifically, e.g. a non-target antigen.

### Linkers

The polypeptides of the present disclosure (e.g. chimeric receptor polypeptides) may additionally comprise further amino acids or sequences of amino acids.

The polypeptides may comprise one or more linker sequences between sequences of amino acids. By way of example, a linker sequence may be provided between different domains of a chimeric receptor polypeptide (e.g. the extracellular portion and the transmembrane domain, or between the transmembrane domain and the intracellular portion).

Linker sequences are known to the skilled person, and are described, for example in Chen et al., Adv Drug Deliv Rev (2013) 65 (10): 1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev (2013) 65 (10): 1357-1369. Flexible linker sequences often comprise high proportions of glycine and/or serine residues.

In some embodiments, a linker sequence comprises at least one glycine residue and/or at least one serine residue. In some embodiments, the linker sequence comprises or consists of glycine and serine residues. In some embodiments, the linker sequence has the structure: (GxS)n or (GxS)nGm; wherein G=glycine, S=serine, x=3 or 4, n=2, 3, 4, 5 or 6, and m=0, 1, 2 or 3. In some embodiments, the linker sequence comprises one or more (e.g. 1, 2, 3, 4, 5 or 6) copies (e.g. in tandem) of the sequence motif G₄S. In some embodiments, a linker sequence comprises or consists of (G₄S)₃ or (G₄S)₄. In some embodiments, the linker sequence has a length of 1-2, 1-3, 1-4, 1-5, 1-10, 1-15, 1-20, 1-25, or 1-30 amino acids. In some embodiments, the linker sequence is one or more repetitions of GGSG.

In some embodiments, the linker sequence comprises one or more copies of an amino acid sequence according to SEQ ID NO:92. In some embodiments, the linker sequence comprises at least 1, 2, 3 or 4 copies of an amino acid sequence according to SEQ ID NO:20.

In some embodiments, the linker sequence comprises, or consists of, an amino acid sequence having at least 60%, preferably one of ≥70%, ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 18.

In some embodiments, the linker sequence comprises a cleavage site, e.g. a cleavage site as described hereinbelow.

The polypeptides of the present disclosure may comprise amino acid sequence(s) to facilitate expression, folding, trafficking, processing, purification or detection thereof. For example, chimeric receptor polypeptides of the present disclosure may additionally comprise a sequence of amino acids forming a detectable moiety, e.g. as described hereinbelow.

The polypeptides may additionally comprise a signal peptide (also known as a leader sequence or signal sequence). Signal peptides normally consist of a sequence of 5-30 hydrophobic amino acids, which form a single alpha helix. Secreted proteins and proteins expressed at the cell surface often comprise signal peptides. Signal peptides are known for many proteins, and are recorded in databases such as GenBank, UniProt and Ensembl, and/or can be identified/predicted e.g. using amino acid sequence analysis tools such as SignalP (Petersen et al., 2011 Nature Methods 8:785-786) or Signal-BLAST (Frank and Sippl, 2008 Bioinformatics 24:2172-2176).

The signal peptide may be present at the N-terminus of the chimeric receptor polypeptide, and may be present in the newly-synthesised polypeptide. The signal peptide provides for efficient trafficking of the chimeric receptor polypeptide. Signal peptides are often removed by cleavage, and thus are not comprised in the mature chimeric receptor polypeptide.

Signal peptides are known for many proteins, and are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as SignalP (Petersen et al., 2011 Nature Methods 8:785-786) or Signal-BLAST (Frank and Sippl, 2008 Bioinformatics 24:2172-2176).

In some embodiments, the signal peptide comprises, or consists of, an amino acid sequence having at least 60%, preferably one of ≥70%, ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 1 or SEQ ID NO: 16.

In some embodiments, chimeric receptor polypeptides of the present disclosure comprise a detectable moiety, a.k.a. a marker. In some embodiments, a detectable moiety is provided at the N-terminus and/or C-terminus of the polypeptide.

In some embodiments, a detectable moiety is a fluorescent label, phosphorescent label, luminescent label, immuno-detectable label (e.g. an epitope tag), radiolabel, chemical, nucleic acid or enzymatic label. The chimeric receptor polypeptides may be covalently or non-covalently labelled with the detectable moiety.

Fluorescent labels include e.g. fluorescein, rhodamine, allophycocyanin, eosine and NDB, green fluorescent protein (GFP), enhanced GFP (eGFP), chelates of rare earths such as europium (Eu), terbium (Tb) and samarium (Sm), tetramethyl rhodamine, Texas Red, 4-methyl umbelliferone, 7-amino-4-methyl coumarin, Cy3, and Cy5. Radiolabels include radioisotopes such as Hydrogen³, Sulfur³⁵, Carbon¹⁴, Phosphorus³², Iodine¹²³, Iodine¹²⁵, Iodine¹²⁶, Iodine¹³¹, Iodine¹³³, Bromine⁷⁷, Technetium^{99m}, Indium¹¹¹, Indium^{113m}, Gallium⁶⁷, Gallium⁶⁸, Ruthenium⁹⁵, Ruthenium⁹⁷, Ruthenium¹⁰³, Ruthenium¹⁰⁵, Mercury²⁰⁷, Mercury²⁰³, Rhenium^{99m}, Rhenium¹⁰¹, Rhenium¹⁰⁵, Scandium⁴⁷, Tellurium^{121m}, Tellurium¹²², Tellurium^{125m}, Thulium¹⁶⁵, Thulium¹¹⁶⁷, Thulium¹⁶⁸, Copper⁶⁷, Fluorine¹⁸, Yttrium⁹⁰, Palladium¹⁰⁰, Bismuth²¹⁷ and Antimony²¹¹. Luminescent labels include as radioluminescent, chemiluminescent (e.g. acridinium ester, luminol, isoluminol) and bioluminescent labels. Immuno-detectable labels include haptens, peptides/polypeptides, antibodies, receptors and ligands such as biotin, avidin, streptavidin or digoxigenin. Nucleic acid labels include aptamers.

In some embodiments, the chimeric receptor polypeptide comprises an epitope tag, e.g. a His, (e.g. 6×His), FLAG, c-Myc, StrepTag, haemagglutinin, E, calmodulin-binding protein (CBP), glutathione-s-transferase (GST), maltose-binding protein (MBP), thioredoxin, S-peptide, T7 peptide, SH2 domain, avidin, streptavidin, and haptens (e.g. biotin, digoxigenin, dinitrophenol), optionally at the N- or C-terminus of the chimeric receptor polypeptide.

In some embodiments, the chimeric receptor polypeptide comprises a moiety having a detectable activity, e.g. an enzymatic moiety. Enzymatic moieties include e.g. luciferases, glucose oxidases, galactosidases (e.g. beta-galactosidase), glucuronidases, phosphatases (e.g. alkaline phosphatase), peroxidases (e.g. horseradish peroxidase) and cholinesterases.

In some embodiments, a polypeptide of the present disclosure comprises a fluorescent label. In some embodiments, the polypeptide comprises an eGFP moiety. In some embodiments, the polypeptide comprises an amino acid sequence having at least 60%, preferably one of ≥70%, ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:7.

Chimeric receptor polypeptides of the present disclosure may also comprise one or more cleavage sites. A cleavage site refers to a sequence of amino acids that acts as a substrate for an enzyme capable of cleaving peptide bonds.

Many such cleavage sites are known to, and can be employed by, the person skilled in the art of molecular biology. In some embodiments, the cleavage sequence comprises an autocleavage site. Autocleavage sites include the 2A cleavage sequence from Picornavirus ‘NPGP’, which is cleaved at ‘G/P’. Further autocleavage sites are described e.g. in Kim et al., PLOS ONE (2011) 6: e18556 (hereby incorporated by reference in its entirety), and include e.g. T2A, P2A, E2A and F2A cleavage sites. The amino acid sequences of T2A and E2A cleavage sites are shown in SEQ ID NOs: 6 and 9, respectively.

A cleavage site may be included in a polypeptide according to the present disclosure to provide for removal of a moiety or domain. It might be desirable to remove a given moiety or domain so that it is not comprised in the polypeptide complex formed by the polypeptide. For example, in embodiments of chimeric receptor polypeptides of the present disclosure, a cleavage site (specifically, a T2A cleavage site) is provided upstream of an eGFP moiety, to provide for its removal such that the eGFP moiety is not included in the final chimeric receptor polypeptide. Accordingly, in some embodiments, a chimeric receptor polypeptides according to the present disclosure comprises a cleavage site adjacent to (i.e. in the amino acid sequence of the polypeptide, e.g. immediately upstream or downstream of) a detectable moiety according to the present disclosure.

In some embodiments, a cleavage site according to the present disclosure is a 2A cleavage site, e.g. selected from a T2A, P2A, E2A and F2A cleavage site. In some embodiments, the cleavage site is a T2A cleavage site.

### Particular Exemplary Chimeric Receptor Polypeptides

In some embodiments, a chimeric receptor polypeptide according to the present disclosure comprises or consists of one of the following structures:

Optional signal peptide-optional tag-optional linker-IL23-binding domain-transmembrane domain-intracellular portion comprising the intracellular domains of IL12Rb2-optional linker-optional marker

Schematic representation of some of the chimeric receptor polypeptides of the present invention are provided in FIG. 1B, Fig. C, Fig. D, Fig. E.

In some embodiments, a chimeric receptor polypeptide according to the present disclosure comprises an amino acid sequence having at least 60%, preferably one of ≥70%, ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:36, 37, 62, 63, 64, 65, 66, 38, 39, 40, 41, 42, 43 or 44.

### Nucleic Acids and Vectors

The present disclosure provides a nucleic acid, or a plurality of nucleic acids, encoding a chimeric receptor polypeptide according to the present disclosure. In some embodiments, the nucleic acid(s) comprise or consist of DNA and/or RNA.

A chimeric receptor polypeptide according to the present disclosure may be produced within a cell by translation of RNA encoding chimeric receptor polypeptide. A chimeric receptor polypeptide according to the present disclosure may be produced within a cell by transcription from nucleic acid encoding the chimeric receptor polypeptide, and subsequent translation of the transcribed RNA.

In some embodiments, the nucleic acid(s) may be, or may be comprised/contained in, a vector, or a plurality of vectors. A ‘vector’ as used herein is a nucleic acid molecule used as a vehicle to transfer exogenous nucleic acid into a cell.

Accordingly, the present disclosure also provides a vector, or plurality of vectors, comprising the nucleic acid or plurality of nucleic acids according to the present disclosure. The vector may facilitate delivery of the nucleic acid(s) encoding a chimeric receptor polypeptide according to the present disclosure to a cell. The vector may be an expression vector comprising elements required for expressing a chimeric receptor polypeptide, optionally together with or linked with a CAR molecule. The vector may comprise elements facilitating integration of the nucleic acid(s) into the genomic DNA of cell into which the vector is introduced.

Nucleic acids and vectors according to the present disclosure may be provided in purified or isolated form, i.e. from other nucleic acid, or naturally-occurring biological material.

A vector may be a vector for expression of the nucleic acid in the cell (i.e. an expression vector). Such vectors may include a promoter sequence operably linked to a nucleotide sequence encoding a chimeric receptor polypeptide according to the present disclosure. A vector may also include a termination codon (i.e. 3′ in the nucleotide sequence of the vector to the nucleotide sequence encoding the chimeric receptor polypeptide and expression enhancers. Any suitable vectors, promoters, enhancers and termination codons known in the art may be used to express a peptide or polypeptide from a vector according to the present disclosure.

The term ‘operably linked’ may include the situation where nucleic acid encoding a chimeric receptor polypeptide according to the present disclosure and regulatory nucleic acid sequence(s) (e.g. a promoter and/or enhancers) are covalently linked in such a way as to place the expression of the nucleic acid encoding a chimeric receptor polypeptide under the influence or control of the regulatory nucleic acid sequence(s) (thereby forming an expression cassette). Thus, a regulatory sequence is operably linked to the selected nucleic acid sequence if the regulatory sequence is capable of effecting transcription of the nucleic acid sequence. The resulting transcript(s) may then be translated into the desired polypeptide(s).

Vectors contemplated in connection with the present disclosure include DNA vectors, RNA vectors, plasmids (e.g. conjugative plasmids (e.g. F plasmids), non-conjugative plasmids, R plasmids, col plasmids, episomes), viral vectors (e.g. retroviral vectors, e.g. gammaretroviral vectors (e.g. murine Leukemia virus (MLV)-derived vectors, e.g. SFG vector), lentiviral vectors, adenovirus vectors, adeno-associated virus vectors, vaccinia virus vectors and herpesvirus vectors), transposon-based vectors, and artificial chromosomes (e.g. yeast artificial chromosomes), e.g. as described in Maus et al., Annu Rev Immunol (2014) 32:189-225 and Morgan and Boyerinas, Biomedicines (2016) 4:9, which are both hereby incorporated by reference in their entirety. In some embodiments, a vector according to the present disclosure is a lentiviral vector.

In some embodiments, the vector may be a eukaryotic vector, i.e. a vector comprising the elements necessary for expression of protein from the vector in a eukaryotic cell. In some embodiments, the vector may be a mammalian vector, e.g. comprising a cytomegalovirus (CMV) or SV40 promoter to drive protein expression.

In some embodiments, a nucleic acid/plurality or vector/plurality according to the present disclosure comprises an EF1α promoter.

In some embodiments, a nucleic acid/plurality or vector/plurality according to the present disclosure encodes a chimeric receptor polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to one of SEQ ID NOs: 36, 37, 62, 63, 64, 65, 66, 38, 39, 40, 41, 42, 43 or 44.

Constituent polypeptides of a chimeric receptor polypeptide according to the present disclosure may be encoded by different nucleic acids of a plurality of nucleic acids according to the present disclosure, or by different vectors of a plurality of nucleic acids according to the present disclosure.

In aspects and embodiments of the present disclosure, a nucleic acid, or a plurality of nucleic acids, according to the present disclosure encodes two or more (e.g. 2, 3, 4 or more) chimeric receptor polypeptides according to the present disclosure.

In some embodiments, wherein a nucleic acid/plurality or vector/plurality encodes two or more (e.g. 2, 3, 4 or more) chimeric receptor polypeptide according to the present disclosure, transcription of nucleic acid encoding the two or more chimeric receptor polypeptides is under the control of the same promoter.

In some embodiments, transcription of nucleic acid encoding the two or more chimeric receptor polypeptides is under the control of different promoters.

In some embodiments, the nucleic acid/plurality or vector/plurality is multicistronic (e.g. bicistronic, tricistronic, etc.). That is, in some embodiments the nucleic acid/plurality or vector/plurality vector comprises multiple polypeptide-encoding nucleotide sequences. In some embodiments, nucleic acid encoding two or chimeric receptor polypeptides is provided in different cistrons.

### Cells Comprising/Expressing the Chimeric Receptor Polypeptides of the Disclosure

The present disclosure also provides a cell comprising a r chimeric receptor polypeptide according to the present disclosure, or a nucleic acid/plurality or vector/plurality according to the present disclosure.

It will be appreciated that where cells are referred to herein in the singular (i.e. ‘a/the cell’), pluralities/populations of such cells are also contemplated.

The cell may be a eukaryotic cell, e.g. a mammalian cell. The mammal may be a primate (rhesus, cynomolgous, non-human primate or human) or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate). In preferred embodiments, the cell is a human cell.

In some embodiments, the cell is an immune cell. An immune cell may be a cell of hematopoietic origin, e.g. a neutrophil, eosinophil, basophil, dendritic cell, lymphocyte, or monocyte. A lymphocyte may be e.g. a T cell, B cell, NK cell, NKT cell or innate lymphoid cell (ILC), or a precursor thereof. The immune cell may express CD27, CD28, CD4 and/or CD8. In some embodiments, the immune cell is a T cell, e.g. a CD3+ T cell. In some embodiments, the T cell is a CD3+, CD4+ T cell. In some embodiments, the T cell is a CD3+, CD8+ T cell. In some embodiments, the T cell is a T helper cell (TH cell). In some embodiments, the T cell is a cytotoxic T cell (e.g. a cytotoxic T lymphocyte (CTL)). In some embodiments, the immune cell is a tumor infiltrating lymphocyte (TIL), B cell, a monocyte, a natural killer (NK) cell, a basophil, an eosinophil, a neutrophil, a dendritic cell, a macrophage, a regulatory T cell (Treg), a helper T cell (Th), a cytotoxic T cell (Tctl), an effector T cell, a memory T cell, a Natural Killer T (NKT) cell, or other T cell.

Aspects and embodiments of the present disclosure relate particularly to T cells comprising/expressing chimeric receptor polypeptides according to the present disclosure.

In some aspects and embodiments, a cell according to the present disclosure expresses/presents a chimeric receptor polypeptide according to the present disclosure at the cell surface. That is, the chimeric receptor polypeptide may be present in or at the cell membrane. Cells can be evaluated for surface expression of chimeric receptor polypeptide, e.g. using antibody-based methods such as flow cytometry (e.g. as described in Examples of the present disclosure).

In aspects and embodiments of the present disclosure, a cell according to the present disclosure comprises or expresses a chimeric receptor polypeptide according to the present disclosure. In some aspects and embodiments, a cell according to the present disclosure comprises nucleic acid encoding a chimeric receptor polypeptide according to the present disclosure. In some aspects and embodiments, a cell according to the present disclosure comprises a nucleic acid/plurality or vector/plurality according to the present disclosure.

In aspects and embodiments of the present disclosure, a cell according to the present disclosure comprises or expresses a polypeptide complex according to the present disclosure that binds to a variant Fc domain as described herein.

Dose-dependent IL12 signalling mediated by exposure of the cells of the invention to IL23 can be investigated as described in the Examples herein, e.g. by detecting, qualifying and analysing the downstream molecules in the pathway, such as STAT4 and their phosphorylation status or level. The level of chimeric receptor polypeptide-mediated signalling can also be analysed using reporter-based methods, e.g. methods quantifying the activity of a transcription factor or gene whose expression/activity is upregulated in response to signalling through IL12.

As used herein, ‘expression’ may be gene or protein expression. Gene expression encompasses transcription of DNA to RNA, and can be measured by various means known to those skilled in the art, for example by measuring levels of mRNA by quantitative real-time PCR (qRT-PCR), or using reporter-based methods. Similarly, protein expression can be measured by various methods well known in the art, e.g. antibody-based methods, for example by western blot, immunohistochemistry, immunocytochemistry, flow cytometry, ELISA, ELISPOT, or reporter-based methods.

An immune cell (e.g. a T cell) according to the present disclosure may display cytotoxicity to cells comprising/expressing a variant Fc domain according to the present disclosure. That is, an immune cell (e.g. a T cell) according to the present disclosure may posses the ability to kill cells comprising/expressing a variant Fc domain according to the present disclosure.

Cytotoxicity and cell killing can be investigated, for example, using any of the methods reviewed in Zaritskaya et al., Expert Rev Vaccines (2011), 9 (6): 601-616, hereby incorporated by reference in its entirety. Examples of in vitro assays of cytotoxicity/cell killing assays include release assays such as the 51Cr release assay, the lactate dehydrogenase (LDH) release assay, the 3-(4,5-dimethylthiazol-2-yl)-2,5-diphenyl tetrazolium bromide (MTT) release assay, and the calcein-acetoxymethyl (calcein-AM) release assay. These assays measure cell killing based on the detection of factors released from lysed cells. Cell killing by a given test cell type (e.g. an immune cell (e.g. a T cell) according to the present disclosure) can be analysed e.g. by co-culturing the test cells with the given target cell type (e.g. a cell comprising a variant Fc domain according to the present disclosure), and measuring the number/proportion of viable (i.e. non-lysed)/dead (e.g. lysed) target cells after a suitable period of time. Other suitable assays include the xCELLigence real-time cytolytic in vitro potency assay described in Cerignoli et al., PLOS One. (2018) 13 (3): e0193498 (hereby incorporated by reference in its entirety), and the Incucyte immune cell killing assay, which is employed in the experimental examples of the present disclosure.

The present disclosure also provides methods for producing a cell according to the present disclosure, and the cells obtained or obtainable by such methods.

Methods for producing cells comprising/expressing a polypeptide/polypeptide complex of interest are well known to the skilled person, and generally comprise introducing nucleic acid(s)/vector(s) encoding the polypeptide(s) of interest into the cells.

Such methods may comprise nucleic acid transfer for permanent (i.e. stable) or transient expression of the transferred nucleic acid. In some embodiments, following introduction into a cell nucleic acid(s) encoding the polypeptide(s) of interest may be integrated into or form part of the genomic DNA of the cell. In some embodiments, following introduction into a cell nucleic acid(s) encoding the polypeptide(s) of interest may be maintained extrachromosomally.

Any suitable genetic engineering platform may be used, and include gammaretroviral vectors, lentiviral vectors, adenovirus vectors, DNA transfection, transposon-based gene delivery and RNA transfection, for example as described in Maus et al., Annu Rev Immunol (2014) 32:189-225, hereby incorporated by reference in its entirety. Methods also include those described e.g. in Wang and Rivière Mol Ther Oncolytics. (2016) 3:16015, which is hereby incorporated by reference in its entirety. Suitable methods for introducing nucleic acid(s)/vector(s) into cells include transduction, transfection and electroporation.

Methods for generating/expanding populations of cells comprising/expressing polypeptide(s) of interest in vitro/ex vivo are well known to the skilled person. Suitable culture conditions (i.e. cell culture media, additives, stimulations, temperature, gaseous atmosphere), cell numbers, culture periods and methods for introducing nucleic acid(s)/vector(s) encoding polypeptide(s) of interest into cells, etc. can be determined by reference e.g. to WO 2018/177966 A1. In some embodiments, a cell/population of cells according to the present disclosure is prepared under GMP (good manufacturing practice; e.g. as described in the guidelines for good manufacturing practice published by the European Commission (Volume 4 of ‘The rules governing medicinal products in the European Union’ contains guidance for the interpretation of the principles and guidelines of good manufacturing practices for medicinal products for human and veterinary use laid down in Commission Directives 91/356/EEC, as amended by Directive 2003/94/EC, and 91/412/EEC respectively) conditions.

Conveniently, cultures of cells according to the present disclosure may be maintained at 37° C. in a humidified atmosphere containing 5% CO₂. The cells of cell cultures can be established and/or maintained at any suitable density, as can readily be determined by the skilled person. Cultures can be performed in any vessel suitable for the volume of the culture, e.g. in wells of a cell culture plate, cell culture flasks, a bioreactor, etc. In some embodiments cells are cultured in a bioreactor, e.g. a bioreactor described in Somerville and Dudley, Oncoimmunology (2012) 1 (8): 1435-1437, which is hereby incorporated by reference in its entirety. Immune cells (e.g. T cells) may be activated prior to introduction of nucleic acid(s) encoding the polypeptide(s) of interest. For example, T cells within a population of PBMCs may be non-specifically activated by stimulation in vitro with agonist anti-CD3 and agonist anti-CD28 antibodies, in the presence of IL-2.

Introducing nucleic acid(s) into a cell may comprise transduction, e.g. lentiviral transduction. Transduction of immune cells with viral vectors is described e.g. in Simmons and Alberola-Ila, Methods Mol Biol. (2016) 1323:99-108, which is hereby incorporated by reference in its entirety.

Agents may be employed to enhance the efficiency of transduction. Hexadimethrine bromide (polybrene) is a cationic polymer which is commonly used to improve transduction, through neutralising charge repulsion between virions and sialic acid residues expressed on the cell surface. Other agents commonly used to enhance transduction include e.g. the poloxamer-based agents such as LentiBOOST (Sirion Biotech), Retronectin (Takara), Vectofusin (Miltenyi Biotech) and also SureENTRY (Qiagen) and ViraDuctin (Cell Biolabs). In some embodiments the methods comprise centrifuging the cells into which it is desired to introduce nucleic acid encoding the polypeptide(s) of interest in the presence of cell culture medium comprising viral vector comprising the nucleic acid (referred to in the art as ‘spinfection’).

The methods generally comprise introducing a nucleic acid encoding polypeptide(s) of interest into a cell, and culturing the cell under conditions suitable for expression of the polypeptide(s) of interest by the cell. In some embodiments, the methods comprise culturing immune cells into which nucleic acid encoding polypeptide(s) of interest has been introduced in order to expand their number.

In some embodiments, the methods comprise analysing the cells to confirm successful introduction of the nucleic acid into the cells. In some embodiments, the methods comprise analysing the cells to confirm expression of the polypeptide(s) of interest by the cells (e.g. via evaluation of a detectable entity).

In some embodiments the methods further comprise cells expressing the polypeptide(s) of interest, e.g. from other cells (e.g. cells which do not express the polypeptide(s) of interest). Methods for purifying/isolating immune cells from heterogeneous populations of cells are well known in the art, and may employ e.g. FACS- or MACS-based methods for sorting populations of cells based on the expression of markers of the immune cells. In some embodiments the methods purifying/isolating cells of a particular type, e.g. CD8+ T cells or CTLs expressing the polypeptide(s) of interest.

Modification of a given target nucleic acid can be achieved in a variety of ways known to the skilled person, including modification of the target nucleic acid by homologous recombination, and target nucleic acid editing using site-specific nucleases (SSNs).

Suitable methods may employ targeting by homologous recombination, which is reviewed, for example, in Mortensen Curr Protoc Neurosci. (2007) Chapter 4: Unit 4.29 and Vasquez et al., PNAS 2001, 98 (15): 8403-8410 both of which are hereby incorporated by reference in their entirety. Targeting by homologous recombination involves the exchange of nucleic acid sequence through crossover events guided by homologous sequences. Other suitable techniques include nucleic acid editing using SSNs. Gene editing using SSNs is reviewed e.g. in Eid and Mahfouz, Exp Mol Med. 2016 October; 48 (10): e265, which is hereby incorporated by reference in its entirety. Enzymes capable of creating site-specific double strand breaks (DSBs) can be engineered to introduce DSBs to target nucleic acid sequence(s) of interest. DSBs may be repaired by either error-prone non-homologous end-joining (NHEJ), in which the two ends of the break are rejoined, often with insertion or deletion of nucleotides. Alternatively, DSBs may be repaired by homology-directed repair (HDR), a high-fidelity mechanism in which a DNA template with ends homologous to the break site is supplied and introduced at the site of the DSB.

SSNs capable of being engineered to generate target nucleic acid sequence-specific DSBs include zinc-finger nucleases (ZFNs), transcription activator-like effector nucleases (TALENs) and clustered regularly interspaced palindromic repeats/CRISPR-associated-9 (CRISPR/Cas9) systems. ZFN systems are reviewed e.g. in Umov et al., Nat Rev Genet. (2010) 11 (9): 636-46, which is hereby incorporated by reference in its entirety. ZFNs comprise a programmable Zinc Finger DNA-binding domain and a DNA-cleaving domain (e.g. a FokI endonuclease domain). The DNA-binding domain may be identified by screening a Zinc Finger array capable of binding to the target nucleic acid sequence. TALEN systems are reviewed e.g. in Mahfouz et al., Plant Biotechnol J. (2014) 12 (8): 1006-14, which is hereby incorporated by reference in its entirety. TALENs comprise a programmable DNA-binding TALE domain and a DNA-cleaving domain (e.g. a FokI endonuclease domain). TALEs comprise repeat domains consisting of repeats of 33-39 amino acids, which are identical except for two residues at positions 12 and 13 of each repeat which are repeat variable di-residues (RVDs). Each RVD determines binding of the repeat to a nucleotide in the target DNA sequence according to the following relationship: ‘HD’ binds to C, ‘NI’ binds to A, ‘NG’ binds to T and ‘NN’ or ‘NK’ binds to G (Moscou and Bogdanove, Science (2009) 326 (5959): 1501.). CRISPR/Cas9 and related systems e.g. CRISPR/Cpf1, CRISPR/C2c1, CRISPR/C2c2 and CRISPR/C2c3 are reviewed e.g. in Nakade et al., Bioengineered (2017) 8 (3): 265-273, which is hereby incorporated by reference in its entirety. These systems comprise an endonuclease (e.g. Cas9, Cpf1 etc.) and the single-guide RNA (sgRNA) molecule. The sgRNA can be engineered to target endonuclease activity to nucleic acid sequences of interest.

In some embodiments, modifying nucleic acid (e.g. endogenous nucleic acid) encoding the chimeric receptor polypeptide in accordance with the present disclosure employs a site-specific nuclease (SSN) system targeting nucleic acid encoding the chimeric receptor polypeptide. The SSN system may be a ZFN system, a TALEN system, CRISPR/Cas9 system, a CRISPR/Cpf1 system, a CRISPR/C2c1 system, a CRISPR/C2c2 system or a CRISPR/C2c3 system.

### Compositions

The present disclosure also provides compositions, e.g. pharmaceutical compositions, comprising the polypeptides, nucleic acids, expression vectors and cells described herein.

The polypeptides, nucleic acids, expression vectors and cells described herein (and particularly the nucleic acids, expression vectors and cells described herein) may be formulated as pharmaceutical compositions or medicaments for clinical use and may comprise a pharmaceutically-acceptable carrier, diluent, excipient or adjuvant. In preferred aspects and embodiments, the present disclosure provides a pharmaceutical composition or medicament comprising a cell according to the present disclosure. Thus, the present disclosure also provides a pharmaceutical composition/medicament comprising a polypeptide, nucleic acid/plurality, expression vector/plurality or cell described herein. In preferred embodiments, a pharmaceutical composition/medicament according to the present disclosure comprises a nucleic acid/plurality, expression vector/plurality or cell described herein.

The pharmaceutical compositions/medicaments of the present disclosure may comprise one or more pharmaceutically-acceptable carriers (e.g. liposomes, micelles, microspheres, nanoparticles), diluents/excipients (e.g. starch, cellulose, a cellulose derivative, a polyol, dextrose, maltodextrin, magnesium stearate), adjuvants, fillers, buffers, preservatives (e.g. vitamin A, vitamin E, vitamin C, retinyl palmitate, selenium, cysteine, methionine, citric acid, sodium citrate, methyl paraben, propyl paraben), anti-oxidants (e.g. vitamin A, vitamin E, vitamin C, retinyl palmitate, selenium), lubricants (e.g. magnesium stearate, talc, silica, stearic acid, vegetable stearin), binders (e.g. sucrose, lactose, starch, cellulose, gelatin, polyethylene glycol (PEG), polyvinylpyrrolidone (PVP), xylitol, sorbitol, mannitol), stabilisers, solubilisers, surfactants (e.g., wetting agents), masking agents or colouring agents (e.g. titanium oxide).

The term ‘pharmaceutically-acceptable’ as used herein pertains to compounds, ingredients, materials, compositions, dosage forms, etc., which are, within the scope of sound medical judgment, suitable for use in contact with the tissues of the subject in question (e.g. a human subject) without excessive toxicity, irritation, allergic response, or other problem or complication, commensurate with a reasonable benefit/risk ratio. Each carrier, diluent, excipient, adjuvant, filler, buffer, preservative, anti-oxidant, lubricant, binder, stabiliser, solubiliser, surfactant, masking agent, colouring agent, flavouring agent or sweetening agent of a composition according to the present disclosure must also be ‘acceptable’ in the sense of being compatible with the other ingredients of the formulation. Suitable carriers, diluents, excipients, adjuvants, fillers, buffers, preservatives, anti-oxidants, lubricants, binders, stabilisers, solubilisers, surfactants, masking agents, colouring agents, flavouring agents or sweetening agents can be found in standard pharmaceutical texts, for example, Remington's ‘The Science and Practice of Pharmacy’ (Ed. A. Adejare), 23rd Edition (2020), Academic Press.

Pharmaceutical compositions and medicaments of the present disclosure may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration. In some embodiments, a pharmaceutical composition/medicament may be formulated for administration by injection or infusion, or administration by ingestion.

Suitable formulations may comprise the cell provided in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

In some embodiments, the pharmaceutical compositions/medicament is formulated for injection or infusion, e.g. into a blood vessel, tissue/organ of interest, or a tumour.

The present disclosure also provides methods for the production of pharmaceutically useful compositions, such methods of production may comprise one or more steps selected from:
producing a cell described herein;
isolating/purifying a cell described herein; and/or
mixing a cell described herein with a pharmaceutically-acceptable carrier, adjuvant, excipient or diluent.

For example, a further aspect the present disclosure relates to a method of formulating or producing a medicament or pharmaceutical composition for use in the treatment of a disease/condition (e.g. a disease/condition described herein), the method comprising formulating a pharmaceutical composition or medicament by mixing a cell described herein with a pharmaceutically-acceptable carrier, adjuvant, excipient or diluent.

### Therapeutic and Prophylactic Applications

The articles of the present disclosure find use in therapeutic and prophylactic methods. In particular, a cell according to the present disclosure, e.g. a cell comprising/expressing a chimeric receptor polypeptide according to the present disclosure, finds use in therapeutic and prophylactic methods. Similarly, a composition according to the present disclosure, e.g. a pharmaceutical composition comprising a cell according to the present disclosure, e.g. a cell comprising/expressing a chimeric receptor polypeptide according to the present disclosure finds use in such methods.

Accordingly, the present disclosure provides a cell or composition described herein for use in a method of medical treatment or prophylaxis. Also provided is a cell or composition described herein for use in a method of treating or preventing a disease or condition described herein. Also provided is the use of a cell or composition described herein in the manufacture of a medicament for treating or preventing a disease or condition described herein. Also provided is a method of treating or preventing a disease or condition described herein, comprising administering to a subject a therapeutically- or prophylactically-effective amount of a cell or composition described herein.

The intervention described in the preceding paragraph may be effective to reduce the development or progression of a disease/condition, alleviate the symptoms of a disease/condition or reduce the pathology of a disease/condition. The intervention may be effective to prevent progression of the disease/condition, e.g. to prevent worsening of, or to slow the rate of development of, the disease/condition. In some embodiments, the intervention may lead to an improvement in the disease/condition, e.g. a reduction in the symptoms of the disease/condition or reduction in some other correlate of the severity/activity of the disease/condition. In some embodiments, the intervention may prevent progression/development of the disease/condition a later stage (e.g. a chronic stage or metastasis).

Therapeutic or prophylactic intervention in accordance with the present disclosure generally comprises administering a cell or pharmaceutical composition according to the present disclosure to a subject to which an antigen-binding molecule comprising: (a) an antigen-binding domain that binds to the target antigen, and (b) a variant Fc domain according to the present disclosure, has been or is to be administered.

In particular, use of the cells and compositions according to the present disclosure in methods to treat/prevent diseases/conditions by adoptive cell transfer (ACT) is contemplated.

Adoptive cell transfer generally refers to a process by which cells (e.g. immune cells) are obtained from a subject, typically by drawing a blood sample from which the cells are isolated. The cells are then typically modified and/or expanded, and then administered either to the same subject (in the case of adoptive transfer of autologous/autogeneic cells) or to a different subject (in the case of adoptive transfer of allogeneic cells). The treatment is typically aimed at providing a population of cells with certain desired characteristics to a subject, or increasing the frequency of such cells with such characteristics in that subject. Adoptive transfer may be performed with the aim of introducing a cell or population of cells into a subject, and/or increasing the frequency of a cell or population of cells in a subject.

Adoptive transfer of immune cells is described, for example, in Kalos and June (2013), Immunity 39 (1): 49-60, and Davis et al. (2015), Cancer J. 21 (6): 486-491, both of which are hereby incorporated by reference in their entirety. The skilled person is able to determine appropriate reagents and procedures for adoptive transfer of cells according to the present disclosure, for example by reference to Dai et al., 2016 J Nat Cancer Inst 108 (7): djv439, which is incorporated by reference in its entirety.

The present disclosure provides methods comprising administering cells and compositions according to the present disclosure to a subject.

Administration of the articles of the present disclosure is preferably in a ‘therapeutically-effective’ or ‘prophylactically-effective’ amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's ‘The Science and Practice of Pharmacy’ (ed. A. Adejare), 23rd Edition (2020), Academic Press.

Administration of the articles of the present disclosure may be parenteral, systemic, intravenous, intra-arterial, intramuscular, intracavitary, intrathecal, intraocular, intravitreal, intraconjunctival, subretinal, suprachoroidal, subcutaneous, intradermal, intrathecal, oral, nasal, topical or transdermal. Administration may be by injection or infusion. Administration of the articles of the present disclosure may be intratumoral. In some cases, the articles of the present disclosure may be formulated for targeted delivery to specific cells, a tissue, an organ and/or a tumor.

Multiple doses of an article of the present disclosure may be provided. Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months.

Administration of a cell or composition according to the present disclosure with an antigen-binding molecule described herein to a subject in accordance with the therapeutic and prophylactic intervention described herein may be simultaneous or sequential.

Simultaneous administration refers to administration of (i) a cell or composition according to the present disclosure, and (ii) an antigen-binding molecule described herein together, for example as a pharmaceutical composition containing both agents (i.e. a combined preparation), or immediately after one another, and optionally via the same route of administration, e.g. to the same artery, vein or other blood vessel.

Sequential administration refers to administration of one of (i) a cell or composition according to the present disclosure, and (ii) an antigen-binding molecule described herein, followed after a given time interval by separate administration of the other agent. It is not required that the two agents are administered by the same route, although this is the case in some embodiments. The time interval may be any time interval.

### Subjects

A subject in accordance with the various aspects of the present disclosure may be any animal or human. Therapeutic and prophylactic applications may be in human or animals (veterinary use).

The subject to be administered with an article of the present disclosure (e.g. in accordance with therapeutic or prophylactic intervention) may be a subject in need of such intervention. The subject is preferably mammalian, more preferably human. The subject may be a non-human mammal, but is more preferably human. The subject may be male or female. The subject may be a patient.

A subject may have (e.g. may have been diagnosed with) a disease or condition described herein, may be suspected of having such a disease/condition, or may be at risk of developing/contracting such a disease/condition. In embodiments according to the present disclosure, a subject may be selected for treatment according to the methods based on characterisation for one or more markers of such a disease/condition.

In some embodiments, a subject may be selected for therapeutic or prophylactic intervention as described herein based on the detection of cells/tissue expressing a target antigen (i.e. the target antigen of an antigen-binding molecule to be employed in conjunction with a cell or composition according to the present disclosure), or of cells/tissue overexpressing the target antigen, e.g. in a sample obtained from the subject.

A subject may be an allogeneic or non-autologous subject with respect to an intervention in accordance with the present disclosure. As used herein, where a subject is referred to herein as being ‘allogeneic’ or ‘non-autologous’ with respect to an intervention, the subject is a subject other than the subject from which the cell of the intervention (i.e. the cell to be administered, or the cell of the pharmaceutical composition/medicament to be administered) is derived. A subject to be treated/prevented in accordance with the present disclosure may be genetically non-identical to the subject from which the cell (e.g. the cell of the pharmaceutical composition/medicament) to be administered to the subject is derived. A subject to be treated/prevented in accordance with the present disclosure may comprise MHC/HLA genes encoding MHC/HLA molecules (e.g. MHC class I a and/or MHC class II molecules) that are non-identical to the MHC/HLA molecules (e.g. MHC class I a and/or MHC class II molecules) encoded by the cell (e.g. the cell of the pharmaceutical composition/medicament) to be administered to the subject. A subject to be treated/prevented in accordance with the present disclosure may be HLA-mismatched with respect to the subject from which the cell (e.g. the cell of the pharmaceutical composition/medicament) to be administered to the subject is derived.

The subject to which cells are administered in accordance with the present disclosure may be allogeneic/non-autologous with respect to the source from which the cell (e.g. the cell of the pharmaceutical composition/medicament) to be administered to the subject is derived. The subject to which cells are administered may be a different subject to the subject from which cells are/were obtained for the production of the cell (e.g. the cell of the pharmaceutical composition/medicament) to be administered to the subject. The subject to which the cell is administered may be genetically non-identical to the subject from which the cell (e.g. the cell of the pharmaceutical composition/medicament) to be administered to the subject cells are/were obtained for the production of the cells.

A subject may be an autogeneic/autologous subject with respect to an intervention in accordance with the present disclosure. As used herein, where a subject is referred to herein as being ‘autogeneic’ or ‘autologous’ with respect to an intervention, the subject is the same subject from which the cell of the intervention (i.e. the cell to be administered, or the cell of the pharmaceutical composition/medicament to be administered) is derived. A subject to be treated/prevented in accordance with the present disclosure may be genetically identical to the subject from which the cell (e.g. the cell of the pharmaceutical composition/medicament) to be administered to the subject is derived. A subject to be treated/prevented in accordance with the present disclosure may comprise MHC/HLA genes encoding MHC/HLA molecules (e.g. MHC class I a and/or MHC class II molecules) that are identical to the MHC/HLA molecules (e.g. MHC class I a and/or MHC class II molecules) encoded by the cell (e.g. the cell of the pharmaceutical composition/medicament) to be administered to the subject. A subject to be treated/prevented in accordance with the present disclosure may be HLA-matched with respect to the subject from which the cell (e.g. the cell of the pharmaceutical composition/medicament) to be administered to the subject is derived.

The subject to which cells are administered in accordance with the present disclosure may be autogeneic/autologous with respect to the source from which the cell (e.g. the cell of the pharmaceutical composition/medicament) to be administered to the subject is derived. The subject to which cells are administered may be the same subject as the subject from which cells are/were obtained for the production of the cell (e.g. the cell of the pharmaceutical composition/medicament) to be administered to the subject. The subject to which the cell is administered may be genetically identical to the subject from which the cell (e.g. the cell of the pharmaceutical composition/medicament) to be administered to the subject cells are/were obtained for the production of the cells.

It must be noted that, as used in the specification and the appended claims, the singular forms ‘a,’ ‘an,’ and ‘the’ include plural referents unless the context clearly dictates otherwise. Ranges may be expressed herein as from ‘about’ one particular value, and/or to ‘about’ another particular value. When such a range is expressed, another embodiment includes from the one particular value and/or to the other particular value. Similarly, when values are expressed as approximations, by the use of the antecedent ‘about,’ it will be understood that the particular value forms another embodiment.

Where a nucleic acid sequence is disclosed herein, the reverse complement thereof is also expressly contemplated.

Methods described herein may preferably be performed in vitro. The term ‘in vitro’ is intended to encompass procedures performed with cells in culture whereas the term ‘in vivo’ is intended to encompass procedures with/on intact multi-cellular organisms.

### EXAMPLES

The following are examples of methods and compositions of the invention. It is understood that various other embodiments may be practiced, given the general description provided above.

### Example 1: Materials and Methods

### 1.1 Recombinant DNA/RNA Techniques

Standard methods were used to manipulate DNA as described in Sambrook et al., Molecular cloning: A laboratory manual; Cold Spring Harbor Laboratory Press, Cold Spring Harbor, New York, 1989. The molecular biological reagents were used according to the manufacturers' instructions. General information regarding the nucleotide sequences of human immunoglobulins light and heavy chains is given in: Kabat, E. A. et al., (1991) Sequences of Proteins of Immunological Interest, 5^{th} ed., NIH Publication No. 91-3242.

### 1.2 DNA Sequencing

DNA sequences were determined by double-strand Sanger sequencing.

### 1.3 Gene Synthesis

Desired gene segments were either generated by PCR using appropriate templates or were synthesized by GeneArt AG (Regensburg, Germany) from synthetic oligonucleotides and PCR products by automated gene synthesis. The gene segments flanked by singular restriction endonuclease cleavage sites were cloned into second generation lenti viral vector vectors. The plasmid DNA was purified from transformed bacteria and concentration determined by UV spectroscopy. The DNA sequences of the subcloned gene fragments were confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells. When more than one protein chain was expressed, the coding sequences were separated by DNA encoding P2A/T2A/E2A self-splicing peptides. E2A is derived from equine rhinitis A virus; P2A is derived from porcine teschovirus-1 2A; T2A is derived from those a asigna virus 2A.

1.4 T Cell Isolation from Fresh Healthy Donor Human Blood

Bio-One LeucoSEP™ Polypropylene Tubes (Greiner, #10349081) were prepared with 15 mL Histopaque®-1077 density gradient medium (Merck, #10771-500ML) by centrifugation at 400 g for 5 minutes until the liquid is under the filter-unit. Fresh blood from anonymized healthy donors was mixed in a 1:1 ratio with Dulbecco's phosphate buffered saline (DPBS, Merck #D8537-500ML) and transferred to the LeuSEP™ tubes without disturbing the prepared solution. After centrifugation at 1200 g for 20 minutes (Acceleration: 1; Break: 0), the buffy coat visible as a white layer was separated into a new 50 mL falcon tube using a 10 mL serological pipette. After washing three times with 40 mL DPBS and centrifugation at 780>450>280 g, the enriched cell population was further purified manually, according to the human Pan T cell isolation kit (Miltenyi, #130-094-535). In brief, cell pellets were labeled with Pan T cell Biotin-Antibody, incubated for 5 minutes at 4° C. and further labeled with Pan T cell magnetic MicroBead Cocktail. Following negative selection on a MACS® Manual Separator, the purified Pan T cell population was either cultured, or frozen as 5×10⁶ cells mL⁻¹ aliquots in advanced RPMI 1640 (Fisher Scientific, #12633012)+20% FBS (Sigma-Aldrich)+1× GlutaMAX™ (Fisher Scientific, #35050061)+20% DMSO (Sigma-Aldrich, #D2650-100ML) in dedicated freezing containers at −80° C. for at least 24 hours, then transferred to liquid nitrogen.

### 1.5 Production of IgG-Like Proteins in Expi293F Cells

Antibodies and antibody-like proteins were generated by transient transfection of Expi293F cells. Cells were seeded in Expi293 media (Gibco, #1435101) at a density of 2.5×10⁶ mL⁻¹ Expression vectors and ExpiFectamine (Gibco, ExpiFectamine transfection kit, #13385544) were separately mixed in OptiMEM (Gibco, #11520386). After 5 minutes, both solutions were combined, mixed by pipetting and incubated for 25 minutes at room temperature. Cells were added to the vector/ExpiFectamine solution and incubated for 24 hours at 37° C. in a shaking incubator with a 5% CO₂ atmosphere. One day post transfection, supplements (Enhancer 1+2, ExpiFectamine transfection kit) were added. Cell supernatants were harvested after 4-5 days by centrifugation and subsequent filtration (0.2 μm filter), and proteins were purified from the harvested supernatant by standard methods as indicated below.

### 1.6 Purification of IgG-Like Proteins

Proteins were purified from filtered cell culture supernatants referring to standard protocols. In brief, Fc containing proteins were purified from cell culture supernatants by Protein A-affinity chromatography (equilibration buffer: 20 mM sodium citrate, 20 mM sodium phosphate, pH 7.5; elution buffer: 20 mM sodium citrate, pH 3.0). Elution was achieved at pH 3.0 followed by immediate pH neutralization of the sample. The protein was concentrated by centrifugation (Millipore Amicon® ULTRA-15, #UFC903096), and aggregated protein was separated from monomeric protein by size exclusion chromatography in 20 mM histidine, 140 mM sodium chloride, pH 6.0.

### 1.7 Production of IgG-Like Proteins in CHO K1 Cells

Alternatively, the antibodies and antibody-like proteins described herein were prepared by Evitria using their proprietary vector system with conventional (non-PCR based) cloning techniques and using suspension-adapted CHO K1 cells (originally received from ATCC and adapted to serum-free growth in suspension culture at Evitria). For the production, Evitria used its proprietary, animal-component free and serum-free media (eviGrow and eviMake2) and its proprietary transfection reagent (eviFect). Supernatant was harvested by centrifugation and subsequent filtration (0.2 μm filter) and afterwards purified from the harvested supernatant by standard methods.

### 1.8 Analytics of IgG-Like Proteins

The concentrations of purified proteins were determined by measuring the absorption at 280 nm using the mass extinction coefficient calculated on the basis of the amino acid sequence according to Pace, et al., Protein Science, 1995, 4, 2411-1423. Purity and molecular weight of the proteins were analyzed by CE-SDS in the presence and absence of a reducing agent using a LabChipGXII or LabChip GX Touch (Perkin Elmer). Determination of the aggregate content was performed by HPLC chromatography at 25° C. using analytical size-exclusion column (TSKgel G3000 SW XL or UP-SW3000) equilibrated in running buffer (200 mM KH₂PO₄, 250 mM KCl pH 6.2, 0.02% NaN₃).

### 1.9 Preparation of Virus-Like Particles

Lipofectamine LTX™-based transfection of ~70% confluent Lenti-X™ 293T cells (Takara, #632180) was performed with CAR/CCR encoding transfer vectors as well as packaging vectors pCAG-VSVG and psPAX2 at a 2:1:2 molar ratio (Giry-Laterriere M, et al Methods Mol Biol. 2011; 737:183-209, Myburgh R, et al Mol Ther Nucleic Acids. 2014). As control for every experiment, mock virus-like particles (VLPs) using only the packaging vectors, but no transfer vector, were produced. After 48 hours, the supernatant was collected and centrifuged for 10 minutes at 500 g to remove remaining cells and concentrated 10-fold (Lenti-x-Concentrator, Takara, #631231) by centrifugation and resuspension according to the manufacturer's protocol.

### 1.10 Transduction of Healthy Donor T Cells

T cells were quickly thawed in a 37° C. water bath and washed with 10× (V:V) advanced RPMI 1640 (Fisher Scientific, #12633012)+10% FBS (Sigma-Aldrich)+1× GlutaMAX™ (Fisher Scientific, #35050061)+50 IU×mL⁻¹ Interleukin-2 (Miltenyi, #130-097-748)+25 ng×mL⁻¹ Interleukin-7 (Miltenyi, #130-095-364)+50 ng×mL⁻¹ Interleukin-15 (Miltenyi, #130-095-766). Cells were then seeded at 10⁶×mL⁻¹ cells in a 12-well plate (Sigma Aldrich, #Z707791-126EA) and activated using Immunocult CD3/CD28/CD2 T cell Activator cocktail (Stemcell Technologies, #10990) for 24 hours.

After brief incubation with 8 μg×mL⁻¹ Polybrene (Sigma Aldrich) and Lentiboost P (1:100) (Sirion Biotech, #SB-P-LV-101-12), previously purified virus-like particles were added to the activated T cells. Following incubation for at least 72 hours, the transduction efficiency was assessed by flow cytometry using fluorescently labeled anti-CAR antigens or commercial fluorescently labeled anti-tag antibodies.

### 1.11 Cultivation of T Cells

Engineered or wild type T cells are cultured in G-Rex® 24 multi-well cell culture plates (Wilson Wolf, #80192M) at densities of 0.2×10⁶ mL⁻¹ to 4×10⁶ ML⁻¹ in advanced RPMI 1640 (Fisher Scientific, #12633012)+10% FBS (Sigma-Aldrich)+1× GlutaMAX™ (Fisher Scientific, #35050061)+50 IU×mL⁻¹ Interleukin-2 (Miltenyi, #130-097-748)+25 ng×mL⁻¹ Interleukin-7 (Miltenyi, #130-095-364)+50 ng×mL⁻¹ Interleukin-15 (Miltenyi, #130-095-766).

### 1.12 Flow Cytometry Surface Staining

Transduction efficiency or cellular signaling events were assessed by flow cytometry. To control transduction efficiency, either GFP was co-expressed via 2A self-cleaving peptide, constructs were tagged with FLAG epitope tags, or fluorescently-labeled antigens were used. Signaling events were assessed via alternate regulation of surface markers. 100,000 cells were prepared in a 96-well plate and stimulated with dilutions of cytokines IL12 (Recombinant Human IL12 (linked heterodimer) Protein, R&D Systems #10018-IL-050), or IL23 (Human IL23 Recombinant Protein, Fisher Scientific #PHC9321). After incubation for 24 hours at 37° C. in a 5% CO₂ atmosphere incubator, cells were washed with DPBS by subsequent centrifugation at 300 g and stained with 50 μL of pre-diluted antibody solution at 4° C. for 30 minutes. Cells were washed two more times before being fixated with 4% PFA solution (Fixation Buffer, BD Biosciences, #554655). Cells were washed once more before final resuspension in FACS-buffer (PBS containing 2% FBS, 10% 0.5 M EDTA, pH 8 and 0.5 g×L⁻¹ NaN₃) and analysed on a BD LSRFortessa™ Cell Analyzer.

### 1.13 Phospho-STAT Staining

In preparation of the assay, 200,000 cells were washed with advanced RPMI 1640 (Fisher Scientific, #12633012)+10% FBS (Sigma-Aldrich)+1× GlutaMAX™ (Fisher Scientific, #35050061) without added cytokines and starved for 4-12 hours in the same medium in a 96 well plate. The cells were stimulated with serial dilutions of cytokines and incubated for 40 minutes at 37° C. in a 5% CO₂ atmosphere incubator. Immediately after, cells were fixated and permeabilized (BD Phosflow™ Fix Buffer I, BD Biosciences, #557870) for 15 minutes at 37° C. Following centrifugation at 300 g for 2 minutes, the cells were resuspended in Phosflow Perm Buffer III (BD Biosciences, 558050) and incubated at 4° C. for 30 minutes. The cells were washed 2 more times in DPBS before being stained with pre-diluted antibody solution using a murine anti-pSTAT4 antibody conjugated with Alexa Fluor™ 647 (BD, #558137) and murine anti-pSTAT3 antibody conjugated with PE (BD, #562072) for 60 minutes at 4° C. After washing the cells once more in FACS-buffer (PBS containing 2% FBS, 10% 0.5 M EDTA, pH 8 and 0.5 g×L⁻¹ NaN₃), the acquisition was done on a BD LSRFortessa™ Cell Analyzer.

### 1.14 Homogeneous Time Resolved Fluorescence

Homogeneous Time Resolved Fluorescence assay was used as an alternative to ELISA in assessing secreted cytokine concentrations. Human IFN gamma kit (Perkin Elmer CisBio, #62HIIFNGPET) was used according to the manufacturer's protocol to determine secreted Interferon gamma concentration in cell culture supernatants. In brief, 100,000 cells were washed with advanced RPMI 1640 (Fisher Scientific, #12633012)+10% FBS (Sigma-Aldrich)+1× GlutaMAX™ (Fisher Scientific, #35050061)+50 IU mL⁻¹ Interleukin-2 (Miltenyi, #130-097-748)+25 ng×mL⁻¹ Interleukin-7 (Miltenyi, #130-095-364)+50 ng×mL⁻¹ Interleukin-15 (Miltenyi, #130-095-766). The cells were stimulated with serial dilutions of IL12 and IL23 and incubated for 24 hours at 37° C. in a 5% CO₂ atmosphere incubator. IFNγ EU Cryptate- and IFNγ XL antibodies were diluted 20-fold in 20× in detection buffer. Next, supernatants were combined 4:1 with a pre-mixed 1:1 dilution of donor and acceptor antibodies and incubated for 4 to 12 hours at room temperature. A standard was acquired as serial 1:3 dilutions from 4000 pg×mL⁻¹ to 0 pg×mL⁻¹. FRET-Fluorescence emission was determined at 665 nm (acceptor) and 620 nm (donor). The data was normalized to the donor signal, background subtracted and fitted to the IFNγ standard curve obtained in the same assay.

### 1.15 Incucyte Killing Assay

Target cells (MKN-45-NLR or HPAF-II-NLR) were seeded in RPMI 1640 (Fisher Scientific, #11875093)+2% FBS (Sigma-Aldrich)+1× GlutaMAX™ (Fisher Scientific, #35050061) in a 96-well plate with edge reservoir (Fisher Scientific, #167425) and incubated for at least 2 hours. Engineered cells were washed two times in Dulbecco's phosphate buffered saline (DPBS, Merck #D8537-500ML), resuspended in RPMI 1640 (Fisher Scientific, #11875093)+2% FBS (Sigma-Aldrich)+1× GlutaMAX™ (Fisher Scientific, #35050061). Cell densities were normalized to equal percentage of CAR-positive cells before being added to the target cells. Engineered T cells were either used at different effector to target cells ratios (E:T) for direct CARs, or at fixed E:T with dilutions of the targeting IgG for adaptor-based CARs. Target cell killing was monitored for 7 days in an Incucyte® Live-Cell Analysis System. Analysis was cut off, when the growth of target cells-only reached a plateau. Data was normalized to the initial cell concentration and to the growth of target cells without effector cells.

In case of a repetitive assay setting (e.g. as in Example 9), target cell killing was monitored for 2 days in an Incucyte® Live-Cell Analysis System per round, indicated by dotted lines. RPMI1640 (Fisher Scientific, #11875093) supplemented with 1× GlutaMAX™ (Fisher Scientific, #35050061) and 10% FBS (Sigma-Aldrich) was chosen as a culture medium. Engineered T cells were washed two times in PBS and normalized to the amount of CAR positive cells by addition of wild-type cells before being added to the target cells at an ET ratio of 1:1. Finally, IL12 or IL23 were added as indicated. After 2 days, corresponding to one round, new target cells were seeded in a new plate. Also, engineered T cells were separated from cancer cells by lightly resuspending and aspirating. Supernatant was removed and fresh medium was added before combining the engineered T cells with the new target cells. Finally, IL12 or IL23 were added again, as indicated. Analysis was continued in the same Incucyte® Live-Cell Analysis System. This process was repeated 4 times for a total of five repetitive killing rounds. Data was normalized to the initial cell concentration within each round.

### Example 2: Detection of IL23-IL12 Chimeric Cytokine Receptors in Human Immune Cells

T cells isolated from 50 mL of healthy donor blood were transduced with VLPs containing transgenes encoding different IL23-IL12 chimeric cytokine receptors (IL23-IL12sR). First, peripheral blood mononuclear cells (PBMCs) were isolated from donated fresh human blood by density gradient centrifugation. The cell population was further enriched for untouched human Pan T cells by negative selection using a human Pan T cell isolation Kit (Miltenyi, #130-096-535). Cells were then cultured for 24 hours in advanced RPMI 1640+10% FBS+1× GlutaMAX+50 IU×mL⁻¹ Interleukin-2+25 ng×mL⁻¹ Interleukin-7+50 ng×mL⁻¹ Interleukin-15, before being stimulated with Immunocult CD3/CD28/CD2 T cell Activator cocktail (StemCELL technologies, #10990) to induce clonal expansion. Previously prepared, 10× concentrated VLPs encoding desired constructs were added together with polybrene (Sigma, #TR-1003-G) and LentiBoostP (Sirion Biotech, #SB-A-LF-901-01) to the cells for subsequent incubation for at least 72 hours. Expression of the full length construct was assessed by eGFP reporter fluorescence, fluorescently labeled anti-CAR antigen (e.g. Biot-Avi-tag_Fc_huCEACAM5 (A3B3) or Fc_(PGLALA)_AF647). The surface expression of IL23-IL12sR was assessed, where applicable, by flow cytometry directly by an N-terminal FLAG-tag (DYKDDDDK) and commercially available fluorescently labeled anti-FLAG-tag antibody (Biolegend, #637322). More specifically, 100,000 cells per condition were harvested, washed with PBS and seeded in a 96 well V-bottom plate. After staining for 30 minutes in the dark at 4° C. using a 20 nM Fc_(PGLALA)_AF647 and 1:200 dilution of BV421 anti-FLAG-tag Antibody in PBS, samples were washed two more times before being fixated with 4% PFA solution (Fixation Buffer, BD Biosciences, #554655). Cells were washed once more before resuspension in FACS-buffer (PBS containing 2% FBS, 10% 0.5 MEDTA, pH 8 and 0.5 g×L⁻¹ NaN₃) and analysis on a BD LSRFortessa™ Cell Analyzer.

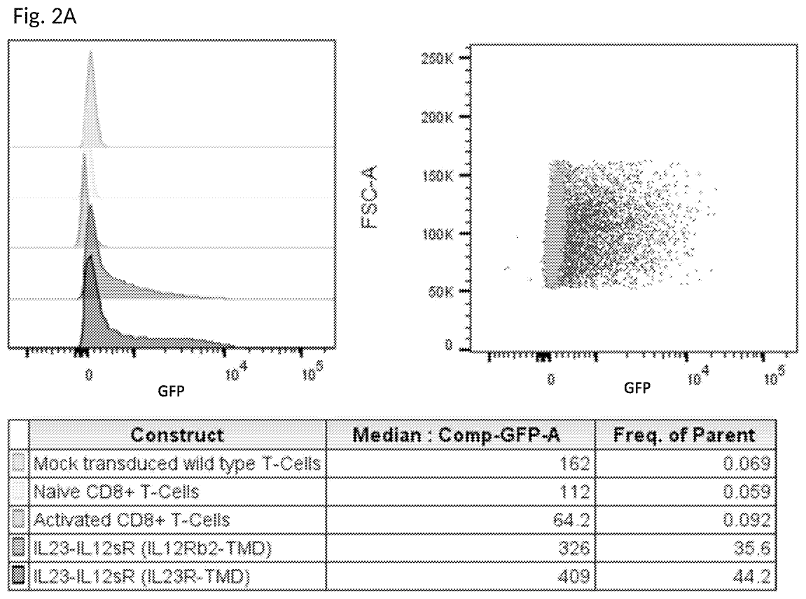

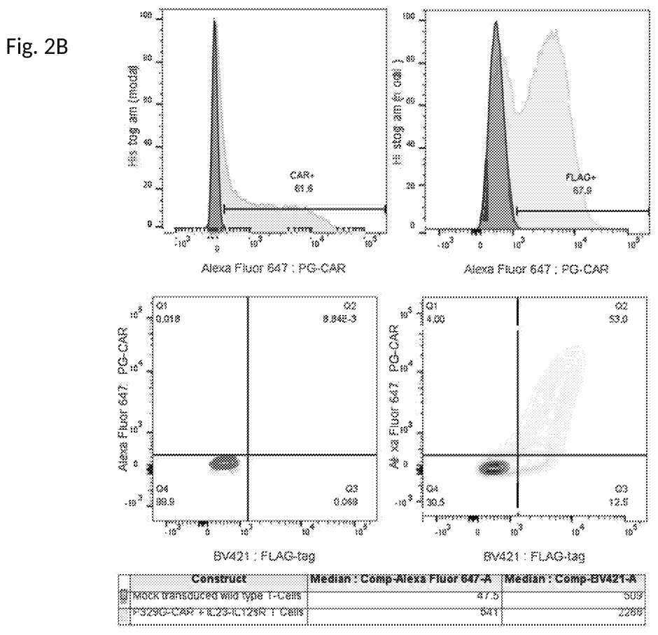

All tested constructs were expressed after transduction of human healthy donor primary T cells, as is indirectly detected by the fluorescent signal of the eGFP reporter protein (FIG. 2A). Furthermore, all recombinant receptors can be directly detected by staining of the N-terminal FLAG-tag (FIG. 2B). Chimeric antigen receptors encoded on the same construct are expressed at comparable ratios, represented by detection via the compatible fluorescently labeled CAR-antigen (FIG. 2B).

### Example 3: Analysis of Signal Transduction and Activation of Transcription Factor Phosphorylation in Human Immune Cells Engineered with an IL23-IL12sR

Analysis of phosphorylation of signal transduction and activators of transcription factors (STATs) by IL23-IL12sR signalling was done by flow cytometric analysis.

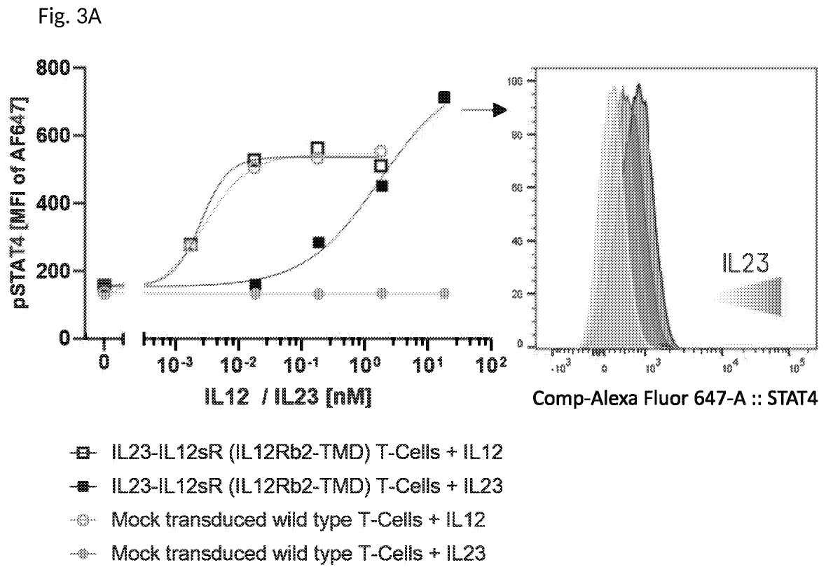

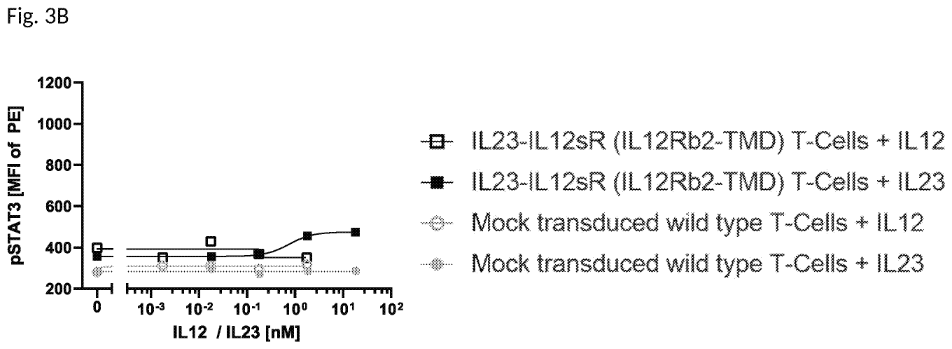

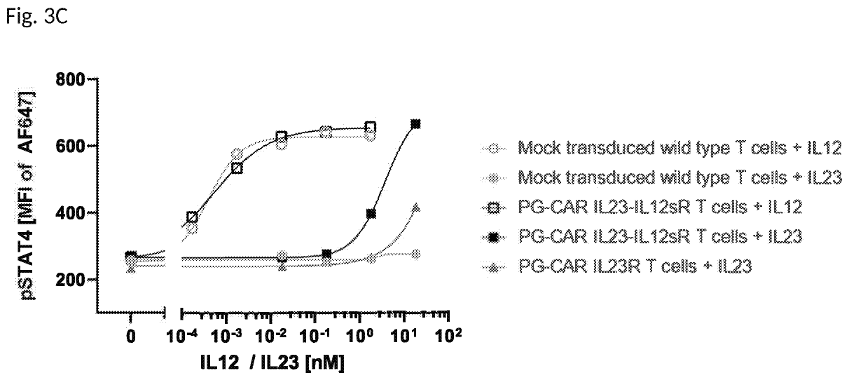

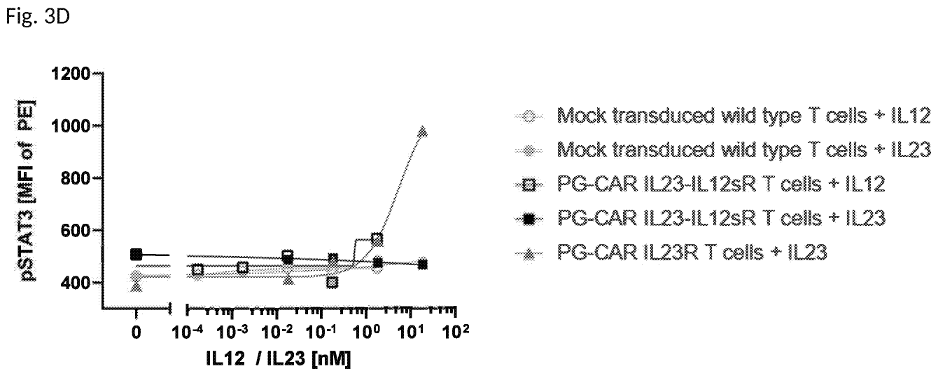

T cells transduced with an IL23-IL12sR were stimulated with serial dilutions of recombinant human interleukin-23 (huIL23) (Thermo Fisher Scientific, #PHC9321) for 40 minutes, followed by immediate fixation (BD, #557870), permeabilization (BD, #558050) and intracellular staining for flow cytometric analysis using a murine anti-pSTAT4 antibody conjugated with Alexa Fluor™ 647 (BD, #558137) and murine anti-pSTAT3 antibody conjugated with PE (BD, #562072). Cells were gated on lymphocytes, single cells (FSC-A vs FSC-H), single cells (SSC-A vs SSC-H) and, where applicable, GFP positive cells. IL23-IL12sR modified cells showed dose-dependent phosphorylation of STAT4 (Jacobson et Al. 1995, Kaplan et Al. 1998) but not STAT3 (Chen et Al. 2003) when stimulated with IL23, similar to the endogenous IL12 receptor complex stimulated with IL12 (FIG. 3A, FIG. 3B). Notably, the EC50 of pSTAT4 upregulation via IL23-IL12sRs is higher and maximum signal appears increased. Combined phosphorylation of STAT4 and STAT3 is only evident in cells where the IL23R is overexpressed (FIG. 3C, Fig. D).

### Example 4: Functional Analysis of IL23-IL12sR-Mediated Upregulation of Characteristic Cell Surface Markers in Engineered Human Immune Cells

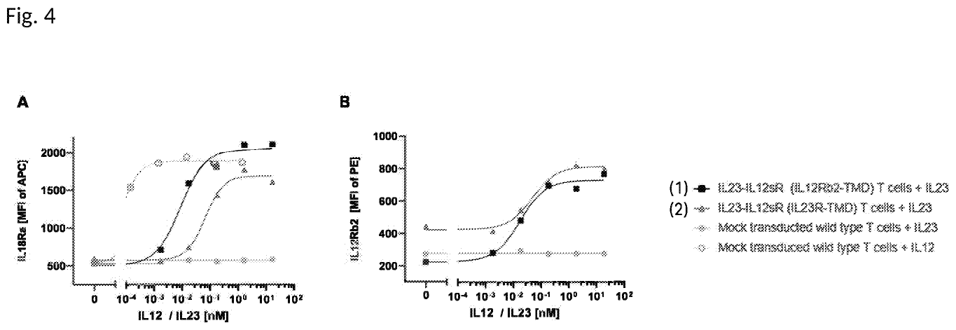

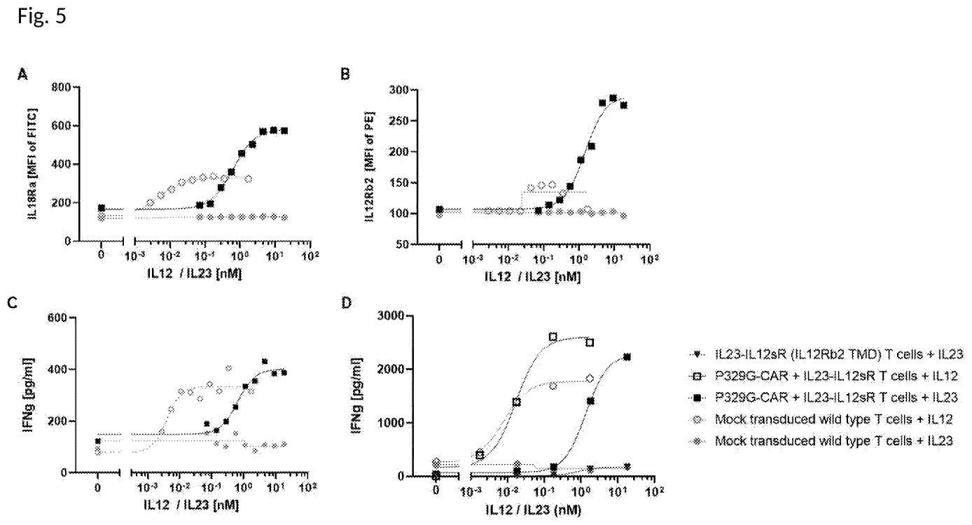

Functional analysis of IL23-IL12sRs was done by flow cytometric analysis of cell surface markers which are known to be expressed upon IL12 signaling. These include most notably IL18Ra (Yoshimoto et Al., 1998), IL12Rb2 via a STAT4 mediated feed-forward loop (Valenzuela et Al., 2002), but also PD-1 (Gerner et Al., 2013) and CD25 (Valenzuela et Al., 2002), although PD-1 and CD25 being not reproducible and donor-dependent, respectively. T cells transduced with an IL23-IL12sR with eGFP reporter were stimulated with serial dilutions of huIL23 (Thermo Fisher Scientific, #PHC9321) for 24 hours. Following incubation, the cells were stained (Const.Nr 1,2: panel 1, Const.Nr 3,4,5,6,7: panel 2, Const.Nr. 8,9: panel 3) and fixated for flow cytometric analysis. The panels are described in Tables 1-3 below. Cells were gated on lymphocytes, singlets (FSC-A vs FSC-H) and living cells expressing CD3 and, where applicable, the GFP reporter or anti-CAR-antigen. The IL23-IL12sR shows dose-dependent upregulation, most prominently of the IL18Ra (FIG. 4A). Here, the EC50 of IL23-IL12sR_(IL12Rb2-TMD) is lowered by approximately 100-fold, and the EC50 of the IL23IL12sR_(IL23R-TMD) by 1000-fold, compared to the signal of IL12 and the endogenous IL12 receptor complex. Similarly, the IL12Rb2 is upregulated by stimulation of the IL23-IL12 chimeric cytokine receptor with IL23 (FIG. 4B), consistent with a previously reported feed-forward mechanisms, triggered by STAT4 signaling (e.g., Valenzuela et Al. 2002). In this example, IL23-IL12sR (IL12Rb2-TMD) T cells show a lower IL12Rb2-baseline, more similar to the wild type cells, when compared to IL23-IL12sR (IL23R-TMD). Specifically, in wild type cells, this feed-forward mechanism cannot be visualized in this manner because the selected anti-IL12Rb2 antibody (Biolegend, #394205) stands in competition with IL12 and is therefore either out-competed or high concentrations of IL12 lead to increased internalization of IL12Rb2. In T cells engineered with a 41BB based P329G CAR in addition to IL23-IL12sR, the upregulation of IL18Ra (FIG. 5A) shows similar dose-response behaviour with a higher EC50 and higher maximum signal. Feed-forward upregulation of IL12Rb2 can also be observed (FIG. 5B).

TABLE 1
Panel 1.

| target | molecule | origin | Cat. Nr. |
|---|---|---|---|
| GFP | n.A. | n.A. | n.A. |
| IL18Ra | APC | Biolegend | 313814 |
| IL12Rb2 | PE | BD Biosciences | 550723 |
| PD1 | BV421 | Biolegend | 329920 |
| CD25 | BV605 | Biolegend | 302631 |
| CD8 | BV711 | Biolegend | 301043 |
| CD3 | BUV395 | BD Biosciences | 740283 |
| Live/DEAD ™ Aqua | Aqua405 | Thermo Fisher | L34957 |
| | | Scientific | |

TABLE 2
Panel 2.

| | | | Cat. |
|---|---|---|---|
| target | molecule | origin | Nr. |
| PG-CAR | Fc_(PGLALA)_AF647 | in-house | |
| IL18Ra | FITC | Biolegend | 313810 |
| IL12Rb2 | PE | BD Biosciences | 550723 |
| PD1 | BV421 | Biolegend | 329920 |
| CD25 | BV605 | Biolegend | 302631 |
| CD8 | BV711 | Biolegend | 301043 |
| CD3 | BUV395 | BD Biosciences | 740283 |
| Live/DEAD ™ | Aqua405 | Thermo Fisher | L34957 |
| Aqua | | Scientific | |

TABLE 3
Panel 3.

| | | | Cat. |
|---|---|---|---|
| target | molecule / fluorophore | origin | Nr. |
| PG-CAR | Biot-Avi- | in-house | |
| | tag_Fc_huCEACAM5(A3 | | |
| | B3) | | |
| Avi-tag | Streptavidin-AF647 | Biolegend | 405237 |
| IL18Ra | FITC | Biolegend | 313810 |
| IL12Rb2 | PE | BD Biosciences | 550723 |
| PD1 | BV421 | Biolegend | 329920 |
| CD25 | BV605 | Biolegend | 302631 |
| CD8 | BV711 | Biolegend | 301043 |
| CD3 | BUV395 | BD Biosciences | 740283 |
| Live/DEAD ™ | Aqua405 | Thermo Fisher | L34957 |
| Aqua | | Scientific | |

### Example 5: Interferon-Gamma Secretion in Response to Stimulation of IL23-IL12sRs in Engineered Human Immune Cells

Upregulation of Interferon-gamma (IFNγ) (Yoshimoto et Al., 1998) in human healthy donor primary T cells transduced with IL23-IL12sRs was assessed by stimulation with serial dilutions of huIL23 (Thermo Fisher Scientific, #PHC9321) for 24 hours. Following incubation cell supernatant was homogenized and transferred to a new vessel. Presence of IFNγ was detected using a HTRF kit (Cis-Bio, #62HIFNGPEG). In brief, supernatants were mixed with IFNγ EU Cryptate- and IFNγ XL antibodies, sealed and incubated for >4 hours at room temperature. The readout was performed by determining the FRET-induced fluorescence emission at 665 nm (acceptor) and fluorescence at 620 nm (donor). Background was subtracted and relative signal was fitted to a standard curve of recombinant IFNγ. The IL23-IL12sR shows dose-dependent upregulation of IFNγ when stimulated with huIL23 (FIG. 5C). Notably, IFNγ release from stimulation of the IL23-IL12sR only occurs, when also a co-stimulating CAR is present (FIG. 5D).

### Example 6: Killing Assay Using Human Immune Cells Engineered with IL23-IL12sR

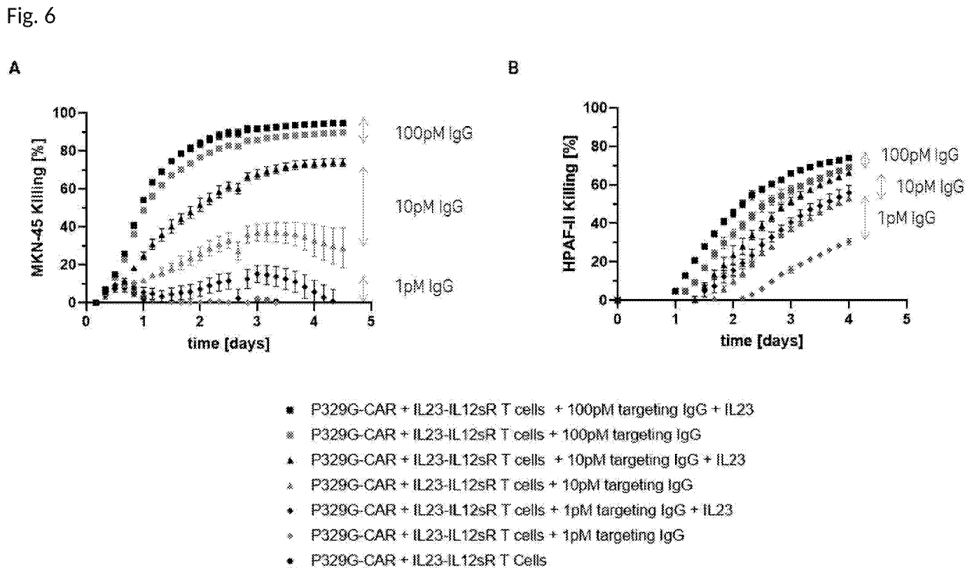

Killing of target cells mediated by IL23-IL12sRs was assessed in the context of adaptor-CAR. Initially, healthy donor human T cells were purified, activated and transduced with VLPs carrying the respective CAR and IL23-IL12sR formats. Cells were expanded for >5 days at 37° C. in a 5% CO₂ atmosphere. Prior to the preparation of the killing assay, transduction efficiency of the immune cells was assessed by flow cytometry, as described in example 1. Target cells MKN-45-NLR or HPAF-II-NLR were seeded in a 96-well plate with edge reservoir and incubated for at least 2 hours to adhere to the plate. Engineered cells were washed two times in PBS and normalized to the amount of CAR positive cells by addition of wild-type cells before being added to the target cells. Engineered T cells were seeded at fixed E:T with dilutions of the targeting IgG for adaptor-based CAR-T cells. Target cell killing was observed for 5-7 days in an Incucyte® live-cell analysis system. Data was normalized to the initial cell concentration and to the growth of target cells without effector cells. In suboptimal killing conditions (e.g., non-saturating concentrations of targeting IgG), the addition of 20 nM of huIL23 to human immune cells engineered with a CAR and IL23-IL12sR, leads to a 20-40% increase in target cell killing (FIG. 6A, FIG. 6B).

### Example 7: Functionality of IL23-IL12sRs in Human Immune Cells Engineered with a Direct CAR

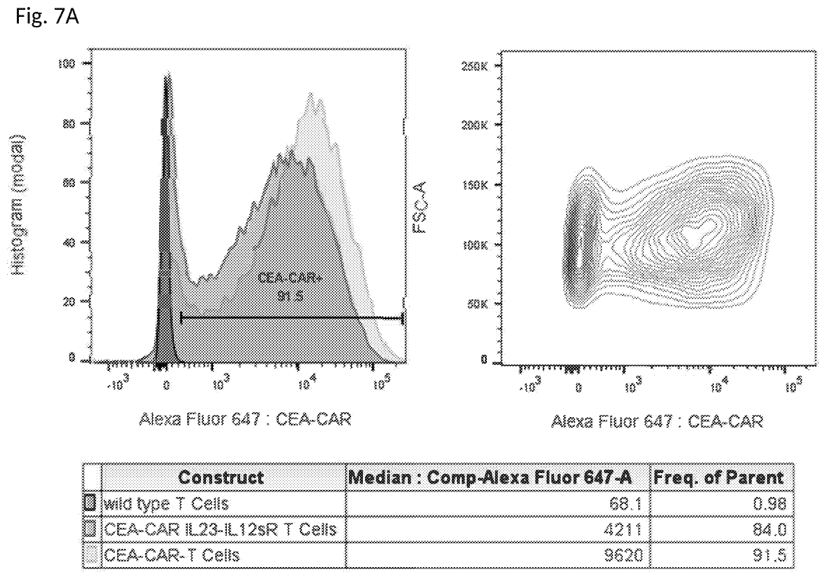

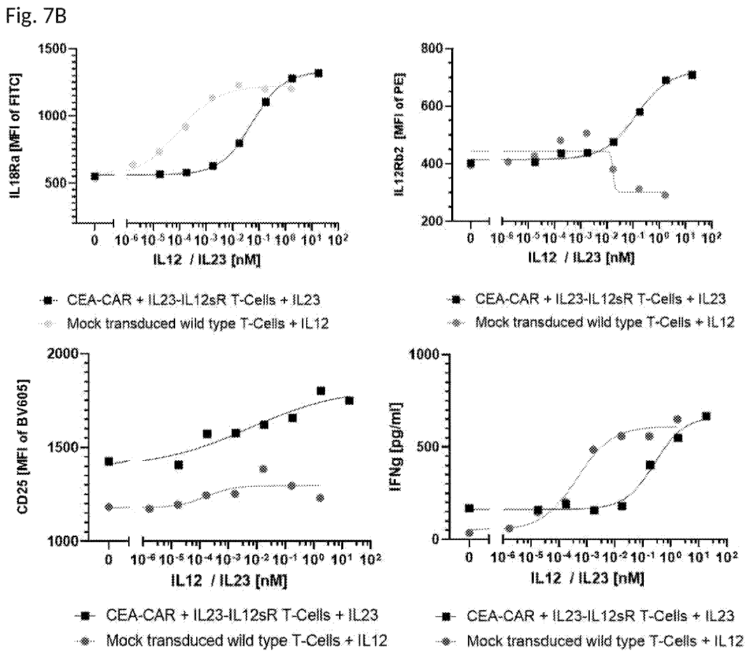

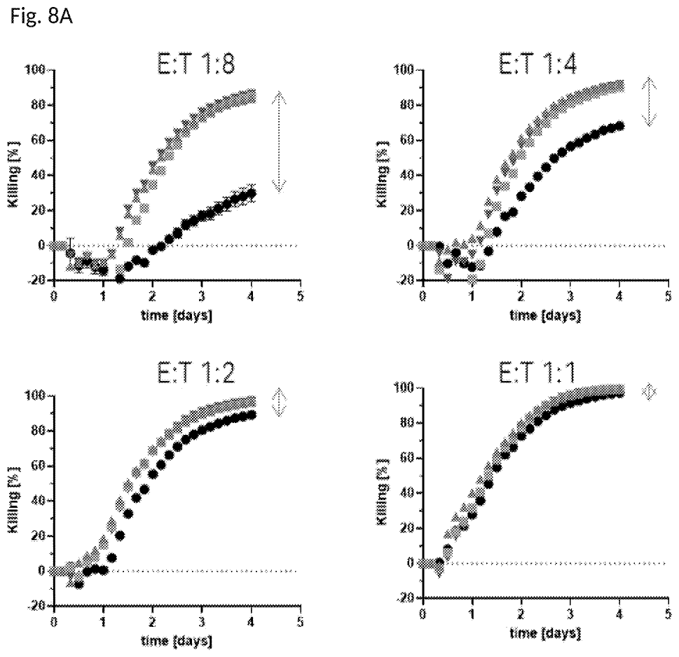

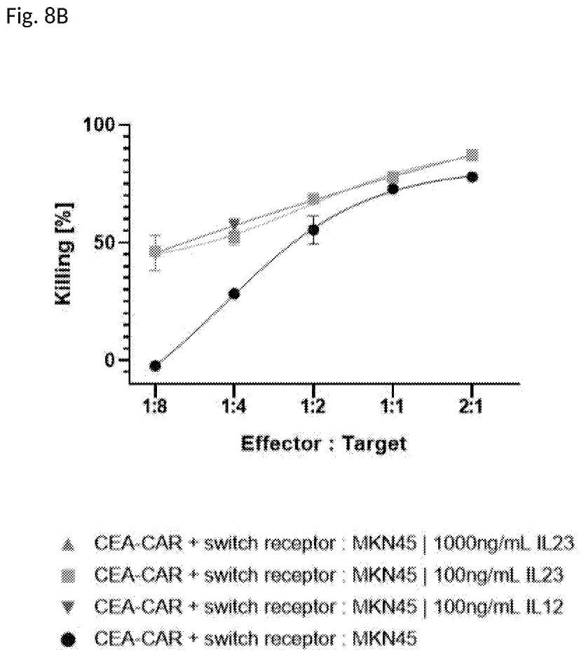

Detection of chimeric constructs, analysis of signal transduction, functional analysis and killing capacity of engineered human immune cells was assessed similar to Examples 2-6. Direct CEA-CAR was detected in flow cytometry using Biot-Avi-tag_Fc_hu_CEACAMS (A3B3) and Streptavidin-AF647 (FIG. 7A). To show that the FLAG-epitope used in other examples does not influence the function of the chimeric receptors, it was omitted from the design in this example. Therefore, the IL23-IL12sRs were only functionally detected via upregulation of IL18Ra, IL12Rb2, CD25 and IFNγ, in accordance with previous assays (FIG. 7B). In suboptimal killing conditions (e.g., low effector to target (E:T) ratios), the addition of 20 nM of huIL23 to human immune cells engineered with a CEA-CAR and IL23-IL12sR, leads to a 10-50% increase in target cell killing (FIG. 8A, FIG. 8B).

### Example 8: IL23-IL12sR Designs with Chimeric ECD Having Similar or Improved Sensitivity and Efficacy when Transduced in Human Immune Cells

Detection of chimeric constructs, analysis of signal transduction and/or functional analysis was assessed similar to Examples 2-7.

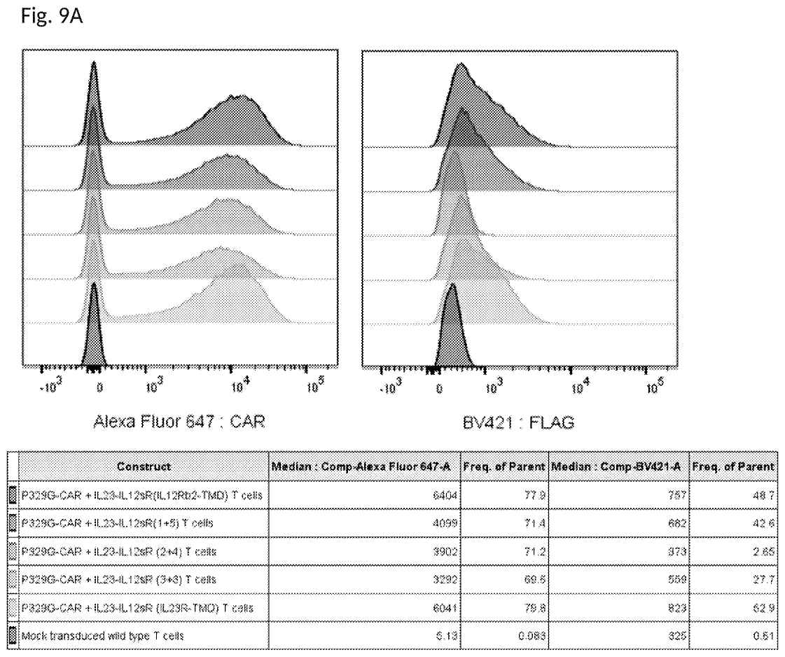

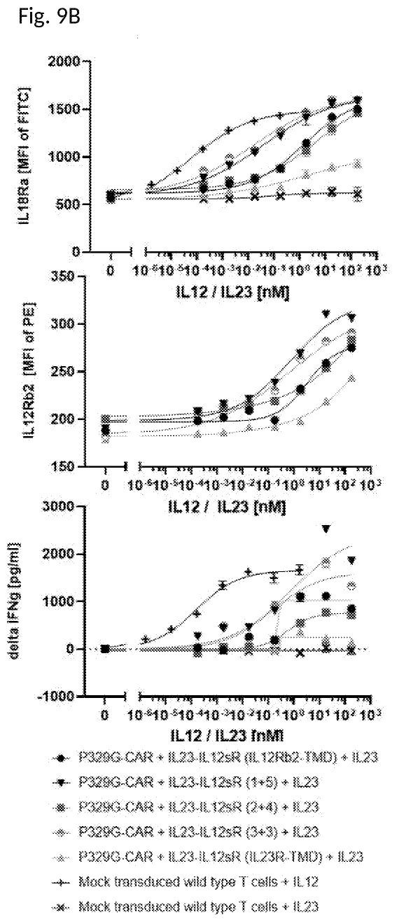

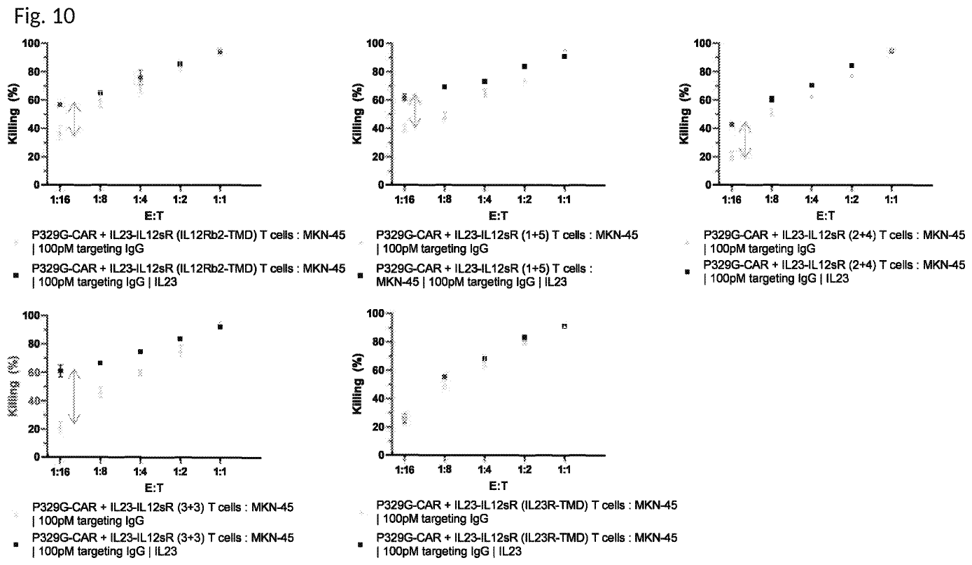

The constructs are expressed after transduction of human healthy donor primary T cells, as is indirectly detected by the expression of the P329G CAR stained with Fc_(PGLALA)_AF647 and directly detected by staining of the FLAG-tag with a fluorescently labeled anti-FLAG-antibody (FIG. 9A). This suggests that IL23-IL12sR designs with a full IL23R-ECD and IL12Rb2-TMD or IL23R-TMD are superior in how they influence the expression of the upstream CAR as well as the expression of the receptor itself, compared to designs with a chimeric ECD. Functionally, stimulation of variants of IL23-IL12sRs with a chimeric ECD1 only, and intact ECD2-3 interface show improved sensitivity and maximum signal across readouts (IL18Ra, IL12Rb2, IFNγ), when stimulated with serial dilutions of IL23 in a dose dependent manner (FIG. 9B). In a target cell killing experiment with MKN-45 cells, all T cells engineered with a P329G CAR-28z and IL23-IL12sR variants except those with IL23-TMD show increased killing capacity of CAR-T cells, especially at low E:T ratios and when stimulated with IL23 (FIG. 10).

### Example 9: Repetitive Killing Assay Using Human Immune Cells Engineered with IL23-IL12sR

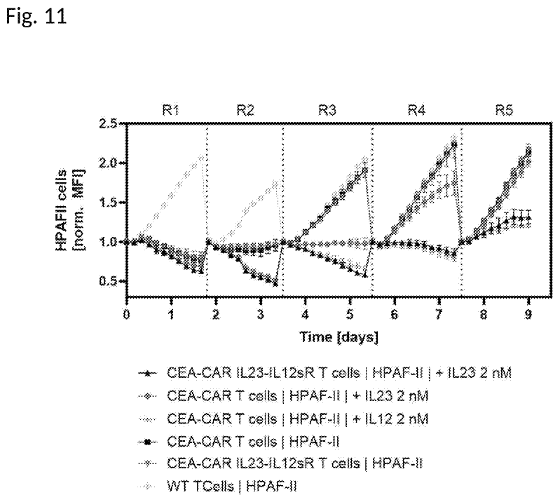

In preparation of the assay, engineered human immune cells were generated and cultured as previously outlined (e.g. under Example 1). Target cells HPAF-II-NLR were seeded in a 96-well plate. In 2-day intervals, engineered T cells (transfected with construct #12 [SEQ ID NO: 66 and 67] or construct #13 [SEQ ID NO: 68 and 6]) were co-cultured with target cells at an ET ratio of 1:1 with or without addition of cytokines and afterwards transferred to new target cells for five repetitive killing rounds. Without the addition of IL12 or IL23, CAR-T cells can control cancer cell growth for 2 rounds (FIG. 11). The killing capacity of both CAR-T cell populations is similar, irrespective of co-engineering with an IL23-IL12sR. By consecutive addition of high-dose IL23 to CAR-T cells, the killing capacity of CAR-T cells is extended. However, only the addition of IL12, or the supplementation of IL23 to CAR IL23-IL12sR T cells confers potent cancer cell killing across five rounds (FIG. 11).

1. A chimeric receptor polypeptide comprising:
(i) an extracellular portion comprising an IL23-binding domain;
(ii) an intracellular portion comprising the intracellular domains of IL12Rb2 (Interleukin-12 receptor beta 2); and
(iii) a transmembrane domain that joins the extracellular portion and the intracellular portion.

2. The chimeric receptor polypeptide of claim 1, wherein the IL23-binding domain binds the p19 subunit of IL23.

3. The chimeric receptor polypeptide of claim 1, wherein the intracellular portion comprises the amino acid sequence of SEQ ID NO: 5 or an amino acid sequence having at least 80% sequence identity thereto.

4. The chimeric receptor polypeptide of claim 1, wherein the transmembrane domain is selected from the transmembrane domain of IL12Rb2, IL23Ra, CD8, and CD28.

5. The chimeric receptor polypeptide of claim 1, wherein the transmembrane domain comprises an amino acid sequence selected from SEQ ID Nos: 3, 4, 22, 33, or an amino acid sequence having at least 80% sequence identity thereto.

6.-11. (canceled)

12. The chimeric receptor polypeptide of claim 1, comprising from the N-terminus to the C-terminus:
signal peptide-tag-linker-IL23-binding domain-transmembrane domain-intracellular portion comprising the intracellular domains of IL12Rb2-linker-marker

13. The chimeric receptor polypeptide of claim 1, wherein the IL23-binding domain comprises an extracellular domain of IL23Ra.

14. The chimeric receptor polypeptide of claim 1, wherein the IL23-binding domain comprises the amino acid sequence of any one of SEQ ID NOs: 10, 35, or 2 or an amino acid sequence having at least 80% sequence identity thereto.

15.-16. (canceled)

17. The chimeric receptor polypeptide of claim 14, further comprising between said IL23-binding domain and the transmembrane domain, an amino acid sequence of any one of SEQ ID NOs: 47, 48, or 15 or an amino acid sequence having at least 80% sequence identity thereto.

18.-19. (canceled)

20. The chimeric receptor polypeptide of claim 1, wherein the IL23-binding domain comprises a Fv, scFv, Fab, Fab′, Fab′-SH, F(ab′)2, crossFab, scFab, a single domain antibody (sdAb), or a designed ankyrin repeat protein (DARPin).

21. The chimeric receptor polypeptide of claim 1, wherein the chimeric receptor polypeptide comprises an amino acid sequence having at least 80% sequence identity to an amino acid sequence selected from SEQ ID Nos: 36-44 and 62-66.

22. A nucleic acid, or a plurality of nucleic acids, encoding a chimeric receptor polypeptide according to claim 1.

23. An expression vector, or a plurality of expression vectors, comprising a nucleic acid or a plurality of nucleic acids according to claim 22.

24. A cell comprising a chimeric receptor polypeptide according to claim 1.

25. The cell of claim 24, wherein the cell expresses IL12Rb1.

26.-29. (canceled)

30. The cell of claim 24, wherein the cell is an immune cell, a neuron, an epithelial cell, an endothelial cell, or a stem cell.

31. (canceled)

32. The cell of claim 30, wherein the immune cell is a tumor infiltrating lymphocyte (TIL), B cell, a monocyte, a natural killer (NK) cell, a basophil, an eosinophil, a neutrophil, a dendritic cell, a macrophage, a regulatory T cell (Treg), a helper T cell (Th), a cytotoxic T cell (Tctl), an effector T cell, a memory T cell, a Natural Killer T (NKT) cell, or other T cell.

33. The cell of claim 32, further comprising a chimeric antigen receptor (CAR) or an engineered TCR.

34. (canceled)

35. The cell of claim 24, wherein the cell shows a dose-dependent phosphorylation of STAT4 but not STAT3 when treated with IL23.

36. (canceled)

37. The cell of claim 24, wherein the cell expresses an antigen binding receptor comprising an anchoring transmembrane domain and an extracellular domain, wherein the extracellular domain comprises an antigen binding moiety comprising
(i) a heavy chain variable domain (VH) comprising a heavy chain complementary determining region (HCDR) 1 of SEQ ID NO:49, a HCDR 2 of SEQ ID NO:50 or SEQ ID NO:51, and a HCDR 3 of SEQ ID NO:52, and
(ii) a light chain variable domain (VL) comprising a light chain complementarity determining region (LCDR) 1 of SEQ ID NO:53, a LCDR 2 of SEQ ID NO:54 and a LCDR 3 of SEQ ID NO:55.

38.-40. (canceled)

41. The cell of claim 37, wherein the antigen binding receptor further comprises at least one stimulatory signaling domain or at least one co-stimulatory signaling domain.

42.-48. (canceled)

49. A pharmaceutical composition comprising the cell of claim 24, and a pharmaceutically acceptable excipient.

50.-52. (canceled)

53. A method for treating a disease, wherein the disease is a cancer, an autoimmune disease, or an infection, the method comprising: administering to a patient the pharmaceutical composition of claim 49.

54. (canceled)

55. A method for modulating the activity of an immune cell, said method comprising: administering, to an immune cell, the nucleic acid or plurality of nucleic acids of claim 22.

56. (canceled)


SEQUENCE LISTING (69 sequences)

SEQ ID NO: 1 - protein, 23 aa, Homo sapiens
MNQVTIQWDAVIALYILFSWCHG

SEQ ID NO: 2 - protein, 332 aa, Homo sapiens
GITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIG

SEQ ID NO: 3 - protein, 21 aa, Homo sapiens
WMAFVAPSICIAIIMVGIFST

SEQ ID NO: 4 - protein, 21 aa, Homo sapiens
LLLGMIVFAVMLSILSLIGIF

SEQ ID NO: 5 - protein, 219 aa, Homo sapiens
HYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 6 - protein, 21 aa, synthetic construct
GSGEGRGSLLTCGDVEENPGP

SEQ ID NO: 7 - protein, 238 aa, synthetic construct
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 8 - protein, 8 aa, synthetic construct
DYKDDDDK

SEQ ID NO: 9 - protein, 23 aa, synthetic construct
GSGQCTNYALLKLAGDVESNPGP

SEQ ID NO: 10 - protein, 100 aa, Homo sapiens
GITNINCSGHIWVEPATIFKMGMNISIYCQAAIKNCQPRKLHFYKNGIKERFQITRINKTTARLWYKNFLEPHASMYCTAECPKHFQETLICGKDISSGY

SEQ ID NO: 11 - protein, 97 aa, Homo sapiens
PPDIPDEVTCVIYEYSGNMTCTWNAGKLTYIDTKYVVHVKSLETEEEQQYLTSSYINISTDSLQGGKKYLVWVQAANALGMEESKQLQIHLDDIVIP

SEQ ID NO: 12 - protein, 96 aa, Homo sapiens
SAAVISRAETINATVPKTIIYWDSQTTIEKVSCEMRYKATTNQTWNVKEFDTNFTYVQQSEFYLEPNIKYVFQVRCQETGKRYWQPWSSLFFHKTP

SEQ ID NO: 13 - protein, 102 aa, Homo sapiens
APEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRP

SEQ ID NO: 14 - protein, 93 aa, Homo sapiens
LPPWDIRIKFQKASVSRCTLYWRDEGLVLLNRLRYRPSNSRLWNMVNVTKAKGRHDLLDLKPFTEYEFQISSKLHLYKGSWSDWSESLRAQTP

SEQ ID NO: 15 - protein, 305 aa, Homo sapiens
EEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKAN

SEQ ID NO: 16 - protein, 19 aa, Homo sapiens
MGWSCIILFLVATATGVHS

SEQ ID NO: 17 - protein, 119 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSS

SEQ ID NO: 18 - protein, 20 aa, synthetic construct
GGGGSGGGGSGGGGSGGGGS

SEQ ID NO: 19 - protein, 109 aa, synthetic construct
QAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVL

SEQ ID NO: 20 - protein, 5 aa, synthetic construct
GGGGS

SEQ ID NO: 21 - protein, 48 aa, Homo sapiens
AKPTTTPAPRPPTPAPTIASQPLSLRPEACRPAAGGAVHTRGLDFACD

SEQ ID NO: 22 - protein, 21 aa, Homo sapiens
IYIWAPLAGTCGVLLLSLVIT

SEQ ID NO: 23 - protein, 42 aa, Homo sapiens
KRGRKKLLYIFKQPFMRPVQTTQEEDGCSCRFPEEEEGGCEL

SEQ ID NO: 24 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 253 aa, Homo sapiens
NRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 26 - protein, 121 aa, synthetic construct
EVQLQQSGAELVEPGASVKLSCTASGFNIKDTYMHWVKQRPEQGLEWIGRIDPANGNSKYVPKFQGKATITADTSSNTAYLQLTSLTSEDTAVYYCAPFGYYVSDYAMAYWGQGTSVTVSS

SEQ ID NO: 27 - protein, 111 aa, synthetic construct
DIVLTQSPASLAVSLGQRATMSCRAGESVDIFGVGFLHWYQQKPGQPPKLLIYRASNLESGIPVRFSGTGSRTDFTLIIDPVEADDVATYYCQQTNEDPYTFGGGTKLEIK

SEQ ID NO: 28 - protein, 218 aa, synthetic construct
DIVLTQSPASLAVSLGQRATMSCRAGESVDIFGVGFLHWYQQKPGQPPKLLIYRASNLESGIPVRFSGTGSRTDFTLIIDPVEADDVATYYCQQTNEDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 450 aa, synthetic construct
EVQLQQSGAELVEPGASVKLSCTASGFNIKDTYMHWVKQRPEQGLEWIGRIDPANGNSKYVPKFQGKATITADTSSNTAYLQLTSLTSEDTAVYYCAPFGYYVSDYAMAYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 222 aa, synthetic construct
CPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 32 - protein, 540 aa, synthetic construct
GLNDIFEAQKIEWHEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGGSQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSAS

SEQ ID NO: 33 - protein, 27 aa, Homo sapiens
FWVLVVVGGVLACYSLLVTVAFIIFWV

SEQ ID NO: 34 - protein, 41 aa, Homo sapiens
RSKRSRLLHSDYMNMTPRRPGPTRKHYQPYAPPRDFAAYRS

SEQ ID NO: 35 - protein, 197 aa, Homo sapiens
GITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIP

SEQ ID NO: 36 - protein, 595 aa, synthetic construct
MNQVTIQWDAVIALYILFSWCHGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIGWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 37 - protein, 854 aa, synthetic construct
MNQVTIQWDAVIALYILFSWCHGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIGLLLGMIVFAVMLSILSLIGIFHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLMLGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 38 - protein, 1398 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGQCTNYALLKLAGDVESNPGPMNQVTIQWDAVIALYILFSWCHGDYKDDDDKGGSGGITNINCSGHIWVEPATIFKMGMNISIYCQAAIKNCQPRKLHFYKNGIKERFQITRINKTTARLWYKNFLEPHASMYCTAECPKHFQETLICGKDISSGYAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 39 - protein, 1393 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGQCTNYALLKLAGDVESNPGPMNQVTIQWDAVIALYILFSWCHGDYKDDDDKGGSGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 40 - protein, 1396 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGQCTNYALLKLAGDVESNPGPMNQVTIQWDAVIALYILFSWCHGDYKDDDDKGGSGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 41 - protein, 1125 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGQCTNYALLKLAGDVESNPGPMNQVTIQWDAVIALYILFSWCHGDYKDDDDKGGSGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSPFFHKTPETVPQVTSKAFQHDTWNSGLTVASISTGHLTSDNRGDIGWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 42 - protein, 1112 aa, synthetic construct
MGWSCIILFLVATATGVHSEVQLQQSGAELVEPGASVKLSCTASGFNIKDTYMHWVKQRPEQGLEWIGRIDPANGNSKYVPKFQGKATITADTSSNTAYLQLTSLTSEDTAVYYCAPFGYYVSDYAMAYWGQGTSVTVSSGGGGSGGGGSGGGGSDIVLTQSPASLAVSLGQRATMSCRAGESVDIFGVGFLHWYQQKPGQPPKLLIYRASNLESGIPVRFSGTGSRTDFTLIIDPVEADDVATYYCQQTNEDPYTFGGGTKLEIKGGGGSAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGQCTNYALLKLAGDVESNPGPMNQVTIQWDAVIALYILFSWCHGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSPFFHKTPETVPQVTSKAFQHDTWNSGLTVASISTGHLTSDNRGDIGWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 43 - protein, 1130 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGQCTNYALLKLAGDVESNPGPMNQVTIQWDAVIALYILFSWCHGDYKDDDDKGGSGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSPFFHKTPETVPQVTSKAFQHDTWNSGLTVASISTGHLTSDNRGDIGWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 44 - protein, 1130 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGQCTNYALLKLAGDVESNPGPMNQVTIQWDAVIALYILFSWCHGDYKDDDDKGGSGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSPFFHKTPETVPQVTSKAFQHDTWNSGLTVASISTGHLTSDNRGDIGLLLGMIVFAVMLSILSLIGIFHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 45 - protein, 1159 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGQCTNYALLKLAGDVESNPGPMNQVTIQWDAVIALYILFSWCHGDYKDDDDKGGSGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSPFFHKTPETVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 46 - protein, 755 aa, synthetic construct
MGWSCIILFLVATATGVHSEVQLQQSGAELVEPGASVKLSCTASGFNIKDTYMHWVKQRPEQGLEWIGRIDPANGNSKYVPKFQGKATITADTSSNTAYLQLTSLTSEDTAVYYCAPFGYYVSDYAMAYWGQGTSVTVSSGGGGSGGGGSGGGGSDIVLTQSPASLAVSLGQRATMSCRAGESVDIFGVGFLHWYQQKPGQPPKLLIYRASNLESGIPVRFSGTGSRTDFTLIIDPVEADDVATYYCQQTNEDPYTFGGGTKLEIKGGGGSAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGQCTNYALLKLAGDVESNPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 47 - protein, 500 aa, Homo sapiens
APEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKAN

SEQ ID NO: 48 - protein, 398 aa, Homo sapiens
LPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKAN

SEQ ID NO: 49 - protein, 5 aa, synthetic construct
RYWMN

SEQ ID NO: 50 - protein, 17 aa, synthetic construct
EITPDSSTINYAPSLKG

SEQ ID NO: 51 - protein, 17 aa, synthetic construct
EITPDSSTINYTPSLKG

SEQ ID NO: 52 - protein, 10 aa, synthetic construct
PYDYGAWFAS

SEQ ID NO: 53 - protein, 14 aa, synthetic construct
RSSTGAVTTSNYAN

SEQ ID NO: 54 - protein, 7 aa, synthetic construct
GTNKRAP

SEQ ID NO: 55 - protein, 9 aa, synthetic construct
ALWYSNHWV

SEQ ID NO: 56 - protein, 119 aa, synthetic construct; identical to SEQ ID NO: 17

SEQ ID NO: 57 - protein, 119 aa, synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSS

SEQ ID NO: 58 - protein, 119 aa, synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSS

SEQ ID NO: 59 - protein, 248 aa, synthetic construct
EVQLVESGGGLVQPGGSLRLSCAASGFTFSRYWMNWVRQAPGKCLEWVGEITPDSSTINYAPSLKGRFTISRDNAKNSLYLQMNSLRAEDTAVYYCARPYDYGAWFASWGQGTLVTVSSGGGGSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGCGTKLTVL

SEQ ID NO: 60 - protein, 109 aa, synthetic construct; identical to SEQ ID NO: 19

SEQ ID NO: 61 - protein, 109 aa, synthetic construct
QAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGCGTKLTVL

SEQ ID NO: 62 - protein, 572 aa, synthetic construct
GITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSPFFHKTPETVPQVTSKAFQHDTWNSGLTVASISTGHLTSDNRGDIGWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 63 - protein, 838 aa, synthetic construct
GITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 64 - protein, 835 aa, synthetic construct
GITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 65 - protein, 840 aa, synthetic construct
GITNINCSGHIWVEPATIFKMGMNISIYCQAAIKNCQPRKLHFYKNGIKERFQITRINKTTARLWYKNFLEPHASMYCTAECPKHFQETLICGKDISSGYAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 66 - protein, 1118 aa, synthetic construct
MGWSCIILFLVATATGVHSEVQLQQSGAELVEPGASVKLSCTASGFNIKDTYMHWVKQRPEQGLEWIGRIDPANGNSKYVPKFQGKATITADTSSNTAYLQLTSLTSEDTAVYYCAPFGYYVSDYAMAYWGQGTSVTVSSGGGGSGGGGSGGGGSDIVLTQSPASLAVSLGQRATMSCRAGESVDIFGVGFLHWYQQKPGQPPKLLIYRASNLESGIPVRFSGTGSRTDFTLIIDPVEADDVATYYCQQTNEDPYTFGGGTKLEIKGGGGSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGQCTNYALLKLAGDVESNPGPMNQVTIQWDAVIALYILFSWCHGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIGWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 67 - DNA, 3357 bp, synthetic construct
  source 1..3357: mol_type = other DNA
atgggctggagctgcatcattctgtttctggtggccacagccacaggcgtgcattctgaagttcagctgcagcagtctggcgccgaacttgttgaacctggcgcctctgtgaagctgtcttgtaccgccagcggcttcaacatcaaggacacctacatgcactgggtcaagcagaggcctgagcagggactcgaatggatcggaagaatcgaccccgccaacggcaacagcaaatacgtgcccaagttccagggcaaagccaccatcacagccgacacaagcagcaacacagcctacctgcagctcaccagcctgacatctgaggacaccgccgtgtactactgcgccccttttggctactacgtgtccgactacgccatggcctattggggccagggcacatctgtgacagtttctagcggaggcggaggaagtggtggcggaggttctggcggcggaggatctgatattgtgctgacacagagccctgccagcctggctgtttctcttggacagagggccaccatgtcttgtagagccggcgagtccgtggatatcttcggcgtgggattcctgcactggtatcagcagaaacccggccagcctcctaagctgctgatctacagagccagcaacctggaatctggcatccctgtgcggtttagcggcaccggcagcagaaccgatttcaccctgatcatcgaccctgtggaagccgatgacgtggccacctactactgccagcagaccaatgaggacccctacacctttggcggaggcaccaagctggaaatcaaaggcggcggaggatccgccaagcctacaacaacaccagctcctagacctccaactcctgctcctacaatcgccagccagcctctgtctctgaggcctgaagcttgtagaccagcagctggcggagccgtgcataccagaggactggatttcgcctgcgacttctgggtgctcgtggttgttggcggagtgctggcctgttactctctgctggtcaccgtggccttcatcatcttttgggtcagaagcaagcggagcagactgctgcacagcgactacatgaacatgacccctagacggcccggacctaccagaaagcactaccagccttacgctcctcctagagacttcgccgcctacaggagcagagtgaagtttagcagatcagccgacgctcctgcctaccagcagggccagaatcagctgtacaacgagctgaacctggggcgcagagaagagtacgacgtgctggataagagaagaggcagagatcccgagatgggcggcaagccccagagaagaaagaatccccaagagggcctctacaacgagctgcaaaaggacaagatggccgaggcctacagcgagatcggaatgaagggcgaacgcagaagaggaaagggccacgacggactgtatcagggcctgtctacagccaccaaggacacctatgatgccctgcacatgcaggccctgcctccaagaggatctggccagtgtaccaactatgccctgctgaagctagccggcgacgttgaatctaatcccgggcctatgaatcaagtgaccatccagtgggatgccgtgatcgccctgtatatcctgttcagctggtgccacggcggcatcaccaacatcaattgctctggccacatctgggtcgagcccgccacaatcttcaagatgggcatgaacatcagcatctactgccaggccgccatcaagaactgccagcctagaaagctgcacttctacaagaacggcatcaaagagcggttccagatcacccggatcaacaagaccaccgccagactgtggtacaagaacttcctggaacctcacgccagcatgtactgcaccgccgagtgtcccaagcacttccaagagacactgatctgcggcaaggacatcagcagcggctaccctccagacatccctgatgaagtgacctgcgtgatctacgagtacagcggcaacatgacctgcacctggaatgccggcaagctgacctacatcgacaccaaatacgtggtgcacgtgaagtccctggaaaccgaggaagaacagcagtacctgaccagcagctacatcaacatctccaccgatagcctgcaaggcggcaagaaatacctcgtgtgggtgcaagccgccaacgctctcggaatggaagagagcaagcagctgcagatccacctggacgacatcgtgattccatctgccgccgtgatcagcagagccgagacaatcaatgccaccgtgcctaagaccatcatctactgggacagccagaccaccatcgagaaggtgtcctgcgagatgagatacaaggccaccaccaaccagacctggaacgtgaaagagttcgataccaacttcacctacgtgcagcagagcgagttctacctcgaaccgaacatcaaatatgtgttccaagtgcggtgccaagaaaccggcaagagatactggcagccttggagcagcctcttcttccacaagacccctgagacagtgccccaagtgacaagcaaggccttccagcacgatacctggaatagcggcctgacagtggccagcattagcaccggtcacctgacctccgacaacagaggcgatatcggatggatggccttcgtggcccctagcatctgtatcgccatcatcatggtcggaatcttcagcacccactacttccagcagaaagtgttcgtgctgttggctgctctgcggccgcagtggtgtagcagagaaatccccgatcctgccaactctacctgcgccaagaagtaccctatcgccgaggaaaagacccagctgcctctggacagactgctgatcgattggcctacacctgaggaccctgagcctctggttatcagcgaagtgctgcaccaagtgacccctgtgttcagacaccctccttgcagcaactggcctcagagagagaagggaatccagggacaccaggccagcgagaaggatatgatgcactctgccagctctccacctcctccaagagcacttcaggccgaaagcagacagctggtggacctgtataaggtgctggaaagcaggggcagcgaccccaagcctgaaaatccagcttgtccttggactgtgctgcctgccggcgatctgcctacacacgatggctacctgcctagcaacatcgacgatctgccatctcatgaggcccctctggccgactctctggaagaactggaaccacagcacatcagcctgagcgtgttccccagtagctctctgcaccctctgaccttcagctgcggcgataagctgaccctggatcagctgaagatgagatgcgacagcctgatgctgtga

SEQ ID NO: 68 - protein, 761 aa, synthetic construct
MGWSCIILFLVATATGVHSEVQLQQSGAELVEPGASVKLSCTASGFNIKDTYMHWVKQRPEQGLEWIGRIDPANGNSKYVPKFQGKATITADTSSNTAYLQLTSLTSEDTAVYYCAPFGYYVSDYAMAYWGQGTSVTVSSGGGGSGGGGSGGGGSDIVLTQSPASLAVSLGQRATMSCRAGESVDIFGVGFLHWYQQKPGQPPKLLIYRASNLESGIPVRFSGTGSRTDFTLIIDPVEADDVATYYCQQTNEDPYTFGGGTKLEIKGGGGSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGQCTNYALLKLAGDVESNPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 69 - DNA, 2286 bp, synthetic construct
  source 1..2286: mol_type = other DNA
atgggctggagctgcatcattctgtttctggtggccacagccacaggcgtgcattctgaagttcagctgcagcagtctggcgccgaacttgttgaacctggcgcctctgtgaagctgtcttgtaccgccagcggcttcaacatcaaggacacctacatgcactgggtcaagcagaggcctgagcagggactcgaatggatcggaagaatcgaccccgccaacggcaacagcaaatacgtgcccaagttccagggcaaagccaccatcacagccgacacaagcagcaacacagcctacctgcagctcaccagcctgacatctgaggacaccgccgtgtactactgcgccccttttggctactacgtgtccgactacgccatggcctattggggccagggcacatctgtgacagtttctagcggaggcggaggaagtggtggcggaggttctggcggcggaggatctgatattgtgctgacacagagccctgccagcctggctgtttctcttggacagagggccaccatgtcttgtagagccggcgagtccgtggatatcttcggcgtgggattcctgcactggtatcagcagaaacccggccagcctcctaagctgctgatctacagagccagcaacctggaatctggcatccctgtgcggtttagcggcaccggcagcagaaccgatttcaccctgatcatcgaccctgtggaagccgatgacgtggccacctactactgccagcagaccaatgaggacccctacacctttggcggaggcaccaagctggaaatcaaaggcggcggaggatccgccaagcctacaacaacaccagctcctagacctccaactcctgctcctacaatcgccagccagcctctgtctctgaggcctgaagcttgtagaccagcagctggcggagccgtgcataccagaggactggatttcgcctgcgacttctgggtgctcgtggttgttggcggagtgctggcctgttactctctgctggtcaccgtggccttcatcatcttttgggtcagaagcaagcggagcagactgctgcacagcgactacatgaacatgacccctagacggcccggacctaccagaaagcactaccagccttacgctcctcctagagacttcgccgcctacaggagcagagtgaagtttagcagatcagccgacgctcctgcctaccagcagggccagaatcagctgtacaacgagctgaacctggggcgcagagaagagtacgacgtgctggataagagaagaggcagagatcccgagatgggcggcaagccccagagaagaaagaatccccaagagggcctctacaacgagctgcaaaaggacaagatggccgaggcctacagcgagatcggaatgaagggcgaacgcagaagaggaaagggccacgacggactgtatcagggcctgtctacagccaccaaggacacctatgatgccctgcacatgcaggccctgcctccaagaggatctggccagtgtaccaactatgccctgctgaagctagccggagatgtggagtctaatcccgggcctgtgtctaaaggcgaggagctgtttaccggagtggtgcccattctggtggagctggatggagatgtgaacggccacaagttcagcgtgtctggcgaaggcgagggagatgccacatatggcaagctgaccctgaagtttatctgcaccaccggcaagctgcccgtgccttggccaacactggtcacaacactgacctacggcgtgcagtgctttagcagataccccgatcacatgaagcagcacgatttcttcaagagcgccatgcctgagggctacgtgcaggagagaaccatcttcttcaaggacgacggcaactacaagaccagagctgaggtgaagttcgagggcgacaccctggtgaacagaatcgagctgaagggaatcgacttcaaggaggatggaaacatcctgggccacaagctggagtacaactacaacagccacaacgtgtacatcatggccgacaagcagaaaaacggcatcaaggtgaacttcaagatccggcacaacatcgaggacggcagcgttcagctggccgaccattaccagcagaatacacccatcggagatggccccgtgctgctgcctgataatcactacctgagcacacagagcgccctgagcaaggaccccaatgagaagagggatcacatggtcctgctggagtttgtgacagccgccggaatcacactcggcatggacgagctgtataagtga